(19) United States
(12) Patent Application Publication
(10) Pub. No.: US 2019/0365290 A1
(43) Pub. Date: Dec. 5, 2019

(54) BODY AND BODY PART ORIENTATION AND POSTURE MONITORING

(71) Applicant: Medtronic, Inc., Minneapolis, MN (US)

(72) Inventors: Brian B. Lee, Golden Valley, MN (US); Patrick Scholten, Hamar (NO)

(21) Appl. No.: 16/109,023

(22) Filed: Aug. 22, 2018

Related U.S. Application Data
(60) Provisional application No. 62/678,841, filed on May 31, 2018.

(51) Int. Cl. A61B 5/11; A61B 5/0205

(52) U.S. Cl. CPC A61B 5/1116; A61B 5/1118; A61B 5/6823; A61B 5/0205; A61B 5/1126

(57) ABSTRACT

Techniques for determining a patient's orientation using an accelerometer that is part of a medical device carried by or implanted in a patient. The medical device may use the accelerometer to determine the orientation of the medical device with respect to gravity. When the medical device is fixed in a known orientation on the patient, the medical device may perform a calculation to determine the orientation of a patient. To simplify calculations and reduce power consumption, the device may calculate an inner product (dot product) multiplication between the orientation vector of the medical device and one or more known reference templates. In some examples, the medical device may determine which reference template is closest to the orientation vector, based on the dot product. In other examples, the medical device may determine one or more body part angles for the patient, based on the dot product.

This application claims the benefit of U.S. Provisional Application 62/678,841 filed May 31, 2018, the entire contents of which is incorporated by reference herein.

### TECHNICAL FIELD

The disclosure relates to body and body part orientation and posture monitoring in medical devices.

### BACKGROUND

Medical devices designed to be worn by or implanted in a patient may automatically monitor a variety of physiologic variables in patients. In some examples, medical devices may monitor physiologic variables over time, such as blood pressure or cardiac rhythm to provide caregivers trend information that may be useful in understanding a patient's health condition in order to provide physicians with evidence as to disease progression or resolution. In other examples, medical devices may automatically adjust therapy based on the measured patient's physical parameters.

Medical devices that include accelerometers that may monitor patient parametrics such as body orientation, posture, activity level, heart rate, heart motion, and sound, including heart sounds, snoring, patient speech, and sound in the patient's environment. Posture sensing may be useful to provide posture trends, such as trends in sleeping orientations, that may help patients, caregivers, and physicians to be aware of recognize and track trends or sudden changes in activities of daily living such as sleep time, sleep quality, and active minutes.

### SUMMARY

In general, the disclosure is directed to techniques for determining a patient's body or body part orientation or posture using an accelerometer that is part of a medical device on or in a patient. In some examples, the medical device may be an implanted medical device. Processing circuitry, e.g. of the medical device, may use the accelerometer to measure patient or limb or whole body activity and the orientation of the medical device relative to gravity.

When the medical device is fixed in a known orientation on or implanted in the patient, the processing circuitry may use the signal(s) from the accelerometer to determine the orientation of the medical device, then perform a calculation to determine the orientation of the part of the body where the device is located and, in some examples, extrapolated to represent the orientation of the entire body. The calculation may include distance metrics such as the dot product to determine the angular distance between the accelerometer indicated orientation vector of the medical device and one or more known measured or calculated accelerometer orientational reference templates stored in a memory. In some examples, the processing circuitry may determine orientation of the body part by determining which of the one or more accelerometer reference orientation templates is closest to the medical device orientation vector, based on the any of a number of possible angular distance metrics, such as the dot product. In some examples, the processing circuitry may characterize overall orientation of the body or body part as one or more body angles, based on the dot product to compare relative body angle to body part orientation references. In some examples, the processing circuitry may also compare the body or body part angles to particular angle thresholds to recognize and characterize standard patient postures like laying on back, standing, laying on left side, etc. In particular, this disclosure describes a method to characterize the orientation of the device relative to the body part efficiently using one upright measured accelerometer reference measurement and in some cases one more laying reference if further refinement of the patient's body or body part orientation is needed.

In one example, the disclosure is directed to a method comprising: storing plurality of reference orientation templates, each of the plurality of reference orientation templates associated with a respective one of a plurality of different orientations of a patient's body part; receiving, by processing circuitry, outputs of a sensor, wherein the sensor is responsive to a gravitational field; determining, by the processing circuitry and based on the outputs of the sensor, an orientation vector of the sensor relative to a gravitational vector of the gravitational field; for each of the plurality of body part orientations, calculating, by the processing circuitry, an angular distance metric between the orientation vector and each of the reference orientation templates; determining, by the processing circuitry, which of the calculated angular distance metrics indicates the associated reference orientation template is closest to the orientation vector; selecting, by the processing circuitry, the one of the plurality of reference orientation templates associated with the closest angular distance metric as the current orientation of the patient's body part.

In another example, the disclosure is directed to a method comprising: storing a first reference orientation template and a second reference orientation template for determining a value representative of a body part angle of a patient, the first reference template orthogonal to the second reference template; receiving, by processing circuitry, outputs of a sensor, wherein the sensor is responsive to a gravitational field; determining, by the processing circuitry and based on the outputs of the sensor, an orientation vector of the sensor relative to a gravitational vector of the gravitational field; selecting, by the processing circuitry, one of the first reference template or the second reference template for determining the orientation value based on the orientation vector; and determining, by the processing circuitry, the value representative of the body part angle of the patient based on a dot product of the orientation vector and the selected one of the first reference template or the second reference template.

In another example, the disclosure is directed to a system comprising: a medical device, wherein the medical device is configured to be attached to a patient's body part in a fixed orientation relative to the patient's body part, the device comprising: a sensor, wherein the sensor is responsive to a gravitational field; processing circuitry operatively coupled to the sensor, wherein the processing circuitry is configured to: receive outputs of the sensor; calculate an orientation vector of the sensor, based on the outputs of the sensor, wherein a direction of the orientation vector is relative to the gravitational field; store a first reference orientation template and a second reference orientation template for determining a value representative of an orientation of the patient's body part, the first reference orientation template orthogonal to the second reference orientation template; select one of the first reference orientation template or the second reference orientation template for determining the value representative of the body angle of the patient's body part based on the orientation vector; and determine the value representative of the body part angle based on a dot product of the orientation vector and the selected one of the first reference orientation template or the second reference orientation template.

This summary is intended to provide an overview of the subject matter described in this disclosure. It is not intended to provide an exclusive or exhaustive explanation of the apparatus and methods described in detail within the accompanying drawings and description below. The details of one or more aspects of the disclosure are set forth in the accompanying drawings and the description below.

### BRIEF DESCRIPTION OF DRAWINGS

FIG. 1 is a conceptual diagram illustrating a medical device system configured to determine patient chest orientation according to one or more techniques of this disclosure.

FIG. 2 is a functional block diagram of an example implantable medical device configured to determine patient body part orientation according to one or more techniques of this disclosure.

FIG. 3 is a functional block diagram of an example external computing device configured to communicate with a medical device.

FIG. 4 is a functional block diagram illustrating an example system that includes external computing devices, such as a server and one or more other computing devices, that are coupled to the medical and the external computing device shown in FIG. 1 via a network.

FIGS. 5A-5C are conceptual diagrams illustrating the roll, pitch and yaw angles of an example medical device which may be worn by or implanted in a patient.

FIG. 6 is a conceptual diagram illustrating an example technique to determine a patient's chest orientation by determining a back-angle and a side-angle.

FIG. 7A is a graph illustrating values of an unscaled dot product for the supine (laying) reference template as a function of values of the orientation vector of a medical device.

FIG. 7B is graph illustrating the relationship between an estimated back-angle determined using an upright reference template and actual measured back-angle values over a range of back-angle values.

FIG. 7C is a graph illustrating the relationship between an estimated back-angle determined using a laying reference template and actual measured back-angle values over a range of back-angle values.

FIG. 7D is a graph illustrating the near linear estimated back-angle versus actual measured back-angle values determined using an upright reference template for the lower part of the range and a laying reference for the upper part of the range.

FIG. 8 is a block diagram illustrating an example technique to determine one or more reference templates by an external computing device and store the reference templates in a medical device.

FIG. 9 is a block diagram illustrating example functions performed to determine patient body part orientation according to one or more techniques of this disclosure.

FIG. 10 is a block diagram illustrating an example technique for determining patient body part orientation using a plurality of reference templates.

FIG. 11 is a block diagram illustrating an example technique for determining patient body part orientation using heuristic classification.

FIG. 12 is a block diagram illustrating an example technique for determining patient body part orientation by determining a scaled back-angle and scaled side-angle.

FIG. 13 is a block diagram illustrating additional details of an example technique for determining a scaled back-angle by a medical device.

FIG. 14 is a block diagram illustrating additional details of an example technique for determining a scaled side-angle by a medical device.

FIG. 15 is a block diagram illustrating an example of a technique to trigger a medical device to update a patient's body part orientation.

### DETAILED DESCRIPTION

The disclosure is directed to techniques for determining a patient's body part orientation using an accelerometer or other orientation sensor that is part of a medical device carried by the patient. In some examples, the medical device may be an implanted medical device. The accelerometer in a medical device may be used to measure general patient or specific body part activity such as limb activity and the orientation of the medical device. When the medical device is fixed in a known or assumed orientation on the patient, the medical device may use the accelerometer to determine the orientation of the medical device relative to gravity, then perform a simple set of calculations to determine the orientation of the part of the patient where the device is implanted, which in many examples may be the chest or abdomen, but may also include head or neck, or limb or foot orientations in some examples.

The calculation may include an angle distance metric, such as a dot product vector multiplication between the accelerometer orientation vector of the medical device and one or more known reference orientation templates stored in a memory location in the medical device. A dot product may also be referred to as an inner product. In some examples, one or more processors may determine body part orientation by determining which of the one or more captured or calculated reference orientation templates is closest to the medical device accelerometer orientation vector, based on the angular distance metric. In some examples, the one or more processors may determine body part orientation based on the dot product, specifically the magnitude and sign of the dot product. In some examples, the one or more processors may perform threshold comparisons on the body part angle or angles to recognize and characterize standard patient body orientations like laying on back, standing, laying on left side, etc. In some examples one or more processors, which may also be referred to as processing circuitry, in the medical device may determine the body part orientation. In some examples, one or more processors external to the medical device, but in communication with the medical device may determine body part orientation, and may perform additional calculations, such as trending, graphs, and similar functions.

In some examples, the calculation to determine a body part orientation of a patient based on the accelerometer orientation vector of the medical device may require the one or more processors to perform complex trigonometric calculations. Complex calculations may require a high-performance processor and the instructions to perform the complex calculations may consume a significant amount of computing energy since the processor may need to be awake for more clock cycles when compared to performing a less computationally intensive referencing and template matching algorithms or dot product angle calculations. The less computationally intensive referencing and template matching algorithms or dot product angle calculations of this disclosure may be considered more efficient than other techniques that may consume more power. More efficient techniques may have advantages, for example for devices powered by a battery. The higher power processor and additional awake time may consume more battery energy, when compared to computing the referencing and template matching or dot product angle calculations, and therefore may be less desirable. Also, in some examples, a small size with a small battery for a medical device may be more desirable, especially for implanted medical devices, because a smaller device may be easier to implant, less invasive and more comfortable, especially for smaller patients.

A device configured to determine a patient's body part orientation using a referencing and template matching algorithm or dot product angle calculations may therefore be configured with a less complex processor that consumes less power and may be awake for less time when compared to a device configured to use more computationally intensive trigonometry calculations. Reduced power consumption may result in longer battery life, and in the example of an implanted medical device, may result in fewer surgeries for the patient to replace the medical device. The referencing and template matching or angle distance metric such as the dot product approaches may therefore have advantages over other techniques for determining a patient's body part orientation.

A dot product, or inner product, calculation between two vectors results in a scalar value that comprises the magnitude of each vector multiplied by the cosine of the angle between them. This may be represented by the below equation:

A·B=∥A∥*∥B∥cos Θ

where Θ is the angle between vectors A and B and ∥A∥ is the magnitude of vector A. By selecting the magnitude of each vector to be one, the dot product is then a scalar value of the cosine of the angle between each vector. Similarly, dividing the dot product by ∥A∥*∥B∥ will result in the scalar value of the cosine of the angle between the vectors. The cosine function is at a maximum when the angle between two vectors is zero. Therefore, the scalar value of the dot product between two 3 axis accelerometer vectors is at a maximum when the angle between the two vectors is zero. A reference template, such as the upright reference, may be a vector that describes the orientation vector a device when the patient is upright. When the device orientation vector is pointing in substantially the same direction as reference template direction, the angle between the orientation vector and the reference template direction is small, i.e. the cosine between the orientation vector and the reference template direction is large. A dot product for the upright reference template that is larger than the dot products for other reference templates (e.g. right side up, face, etc.), may indicate that the patient is closer in angle to upright than the other body orientations.

Determining the cosine between two angles may also be described as an “angle or angular distance calculation” or rather an “inverse angular distance calculation” between two 3-axis accelerometer vectors. The cosine between two accelerometer vectors describes the projection of one accelerometer vector on the other accelerometer vector, i.e. an angular distance. Therefore, when the cosine is large, the angle between the two vectors is small and the projection of one vector on the other is large.

Another example of an angular distance metric calculation for comparison to body reference orientation templates may include measuring the total magnitude of the absolute value of the differences between each of the X, Y and Z values of each vector. For example, when the angle between two vectors is small the difference between the X-values will be small (e.g. |X1−X2|=small). Similarly, the absolute value of the difference between the Y-values and Z-values will be small when the vectors have approximately the same angle and magnitude. Therefore, the angular distance calculation may be considered to be small when the angle is small between accelerometer vectors of the same relative magnitude. Some examples of techniques to calculate this type of angular distance calculation may include the absolute value of the difference or take the square root of the sum of the square of the differences of the X, Y and Z values of each vector.

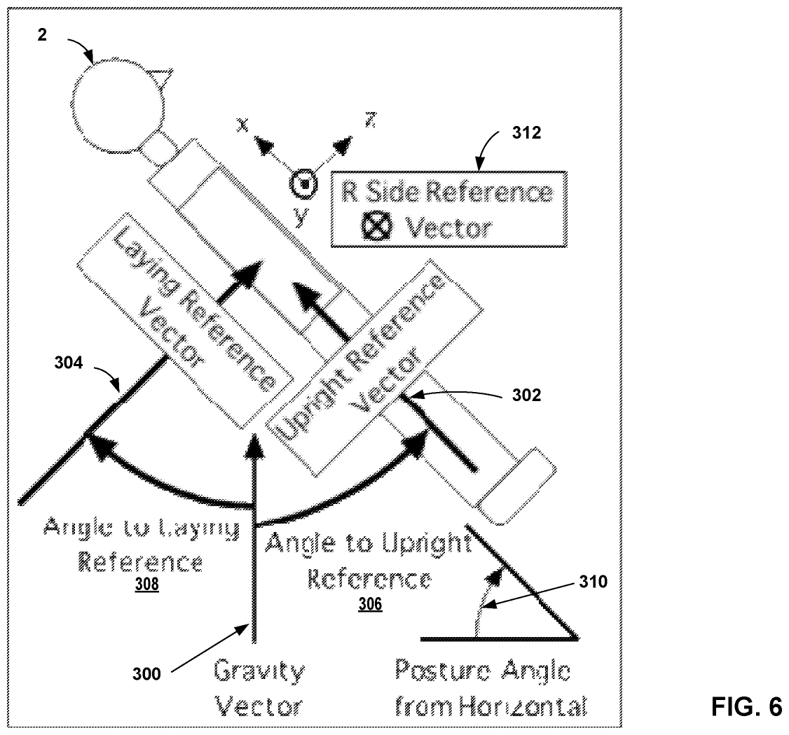

Similarly, a patient's body part orientation may be represented as the patient's body part angle based on the patient's device orientation at any given time relative to one or two, or three orthogonal reference orientation vectors, such as an upright reference vector, a laying reference vector and a side reference vector. One useful body angle or body part angle can be defined as being the angle of the body part relative to the earth's plane. An example of this is depicted in FIG. 6, explained in more detail below. The angle of the cranial to caudal midline of the chest relative to earth's plane, via a 3-axis accelerometer located in the chest, can be measured by determining the angular distance between the device's 3D accelerometer gravity vector, which points to the center of the earth, and one or both of two stored accelerometer reference vectors—the laying reference and the upright reference. In order to measure the rotation angle of the chest around the cranial to caudal midline of the chest, the device's 3D accelerometer gravity vector may be compared to one or both of two stored accelerometer vectors, e.g. a right side up vector running from a patient's left shoulder through the right shoulder, and the laying reference. In other examples, the rotation angle may also use the left side up vector, but with different sign conventions.

A processor may determine the relative angle of the midline of the chest (back-angle) by calculating a dot product between the device accelerometer vector and an upright reference template. The dot product value then represents the cosine of the angle between the midline of the chest and the upright reference. The device may translate the result to be relative to the orthogonal direction of the earth's plane by a 90-degree rotation. In some examples, the processor may directly use the dot product in calculations or other functions, rather than converting the dot product to an angle using the inverse-cosine function. Using the unconverted dot product may have advantages in reducing calculation complexity. In other examples, the dot product may be transmitted to an external device, which may perform additional calculations, such as converting the dot product to an angle.

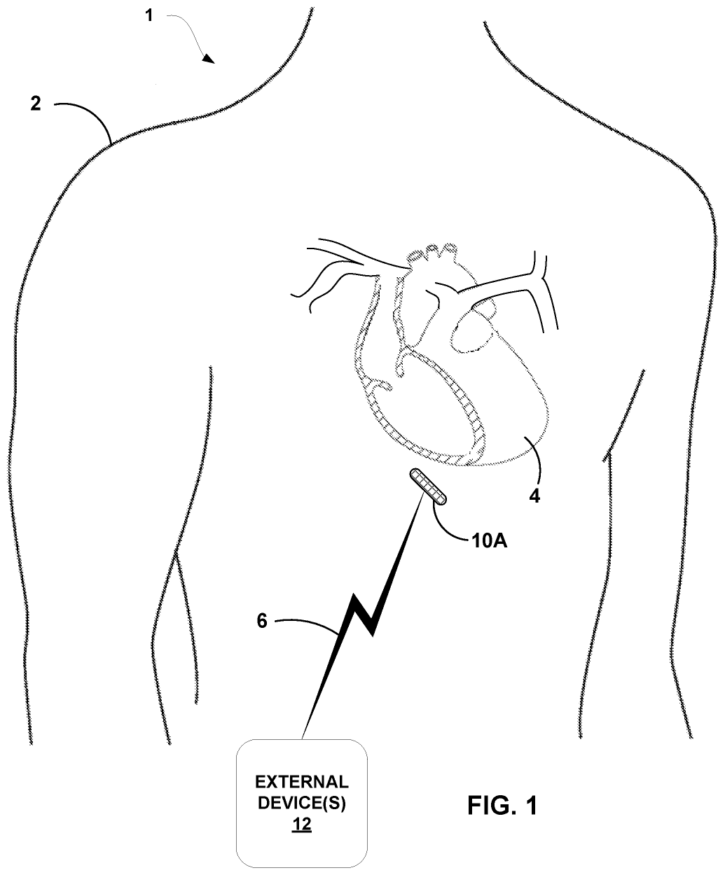

FIG. 1 is a conceptual diagram illustrating a medical device system configured to determine patient chest orientation and posture according to one or more techniques of this disclosure. Example medical system 1 also may be referred to as a “medical system” or a “system”. In general, systems (e.g., system 1) may include one or more medical devices, leads, implanted or external sensors, external devices, or other components configured for techniques described herein. Medical system 1 is an example of a medical device system configured to implement the techniques described herein for monitoring the chest orientation, posture, and other physiological parameters of patient 2, which may include cardiovascular pressure, blood pressure, patient motion, patient activity, or heart rate. In the illustrated example, medical system 1 includes an implantable medical device (IMD) 10 (also may be referred to as an implantable monitoring device, an implantable hub device, or an insertable cardiac monitor (ICM)), such as may be in communication with one or more external devices 12 or one or more other implanted devices, not shown in FIG. 1. Such other implantable devices may be an implantable pacemaker, an implantable cardiac defibrillator, a cardiac resynchronization therapy (CRT) device (e.g., CRT-D defibrillator or CRT-P pacemaker), a neuro-stimulator, a nerve stimulator, a drug pump (e.g., an insulin pump), or other devices. In some examples, some of these devices may alternatively be external medical devices, such as the insulin pump. In some examples another implantable device, also not shown, may include an implantable pressure sensing device that may be implanted within a pulmonary artery of heart 4. The communication link between external device 12 and IMD 10A is indicated by communication link 6.

In the example of FIG. 1, IMD 10A is an insertable cardiac monitor (ICM) capable of sensing and recording electrocardiogram (ECG) signals from a location outside of heart 4 via electrodes. In some examples, IMD 10A includes or is coupled to one or more additional sensors, such as accelerometers, that generate one or more signals that vary based on patient motion or chest orientation, blood flow, impedance, heart rate, oxygen saturation, or respiration. IMD 10A may monitor a physiological parameter indicative of patient state, such as posture, heart rate, activity level, heart rate, or respiration rate, and IMD 10A may measure the one or more physiological parameters at times when another implantable medical device, not shown, is measuring cardiovascular pressure. IMD 10A may include a light emitter and light detector configured to emit a signal (e.g., a light signal) into a target site of patient 2.

IMD 10A may include processing circuitry as well as a sensor, such as a 3-axis accelerometer, that is responsive to a gravitational field. The processing circuitry of IMD 10A may receive the outputs of the sensor and determine the posture of patient 2, based on the outputs of the sensor and the one or more reference templates.

In some examples, IMD 10A may be implanted outside of the thoracic cavity of patient 2 (e.g., subcutaneously or submuscularly, such as the pectoral location). IMD 10A may be positioned near the sternum near or just below the level of heart 4. In some examples, IMD 10A may take the form of a Reveal LINQ™ ICM, available from Medtronic plc, of Dublin, Ireland. In other examples, IMD 10A may take the form of a pacemaker, defibrillator, drug delivery system or other types of medical device.

IMD 10A may include a timer and processing circuitry configured to determine a time of day based on the timer value or may determine the amount of time between events (e.g., posture changes). IMD 10A may include wireless communication circuitry configured to transmit or receive a signal from another device, such as to or from one or more external devices 12, or to another implanted medical device, for example. IMD 10A may determine a patient's posture, for example, and transmit that determination to external device 12. IMD 10A may also configured to receive the one or more reference templates from external device 12 and store the reference templates in a memory location within IMD 10A.

IMD 10A may transmit data about the posture, and other physiological parameter data acquired by IMD 10A, to external device 12. IMD 10A may also transmit cardiovascular pressure measurements, for example, received from another implanted sensor device, not shown, to external device 12, such as when IMD 10A is functioning as a hub device. For example, IMD 10A may transmit any data described herein related to cardiovascular pressure, posture, heart rate, activity level, respiration rate, or other physiological parameters to external device 12. For purposes of this disclosure, a chest posture, or other physiological measurements may include one or more numerical values, multi-variable tables (e.g., such values as a function of time), or other techniques for storing such data.

External device 12 may include processing circuitry configured to communicate, execute programming instructions, provide alerts to a patient or others and perform other functions. In some examples, external device 12 may be a computing device (e.g., used in a home, ambulatory, clinic, or hospital setting) to communicate with IMD 10A via wireless telemetry. External device 12 may include or be coupled to a remote patient monitoring system, such as Carelink®, available from Medtronic plc, of Dublin, Ireland. External device 12 may be, as an example, a programmer, external monitor, or a consumer device (e.g., a smart phone). In some examples, external device 12 may receive data, alerts, patient instructions, or other information from IMD 10A. External device 12 may be used to program commands or operating parameters into IMD 10A for controlling its functioning (e.g., when configured as a programmer for IMD 10A). External device 12 may be used to interrogate IMD 10A to retrieve data, including device operational data as well as physiological data accumulated in IMD memory. The interrogation may be automatic, such as according to a schedule, or in response to a remote or local user command. Programmers, external monitors, and consumer devices are examples of external devices 12 that may be used to interrogate IMD 10A. Examples of communication techniques used by IMD 10A and external device 12 include radiofrequency (RF) telemetry, which may be an RF link established via Bluetooth, WiFi, or medical implant communication service (MICS). In other words, medical system 1 is an example of a medical device system configured to determine, monitor and track the chest orientation, posture, and activity of patient 2. The techniques described herein may be performed by processing circuitry of medical system 1, such as processing circuitry of one or more of IMD 10A, external device 12, and one or more other implanted or external devices not shown, individually, or collectively.

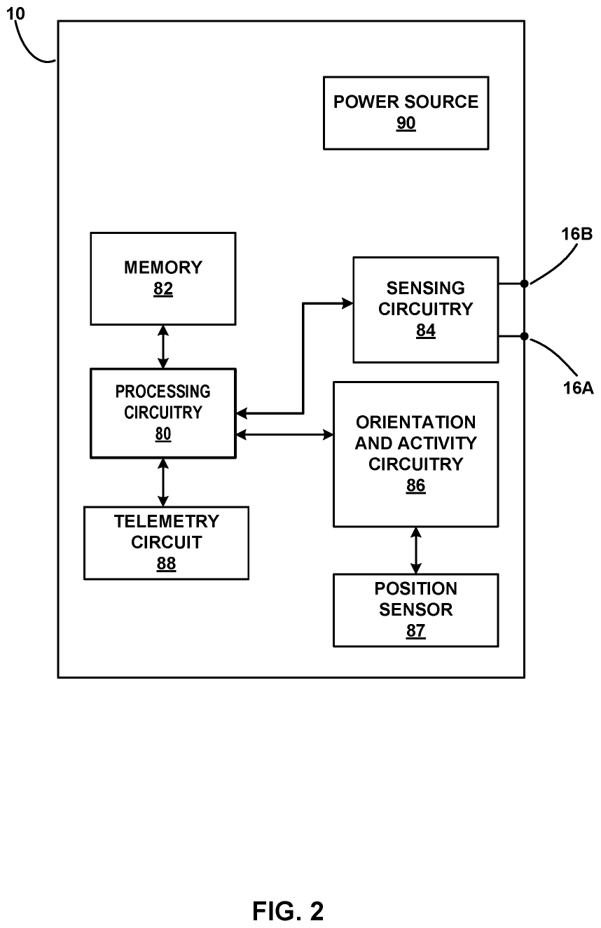

FIG. 2 is a functional block diagram illustrating various components of an IMD 10. IMD 10 of FIG. 2 may include the same functions and features as described above for IMD 10A in relation to FIG. 1. Similarly, the description of IMD 10 below may also apply to other medical devices with orientation and activity state sensing circuitry that may be attached to a patient, such as by straps or other means.

In the example of FIG. 2, IMD 10 includes a processing circuitry 80, memory 82, sensing circuitry 84, orientation and activity circuitry 86, telemetry circuit 88, and power source 90. Memory 82 may store instructions for execution by processing circuitry 80, stimulation therapy data, orientation and activity state information, orientation and activity state indications, and any other information regarding therapy or patient 2, as depicted in FIG. 1. Therapy information may be recorded for long-term storage and retrieval by a user, and the therapy information may include any data created by or stored in IMD 10. Memory 82 may include separate memories for storing instructions, orientation and activity state information, program histories, and any other data that may benefit from separate physical memory modules.

Processing circuitry 80 controls sensing circuitry 84 to sense physiological signals via electrode combinations formed by electrodes in one or more electrode arrays. For example, sensing circuitry 84 may receive signals via electrodes on one or more leads 16A and 16B. Components described as processors, or processing circuitry within IMD 10, external device 12, as described above in relation to FIG. 1, or any other device described in this disclosure may each comprise one or more processors, such as one or more microprocessors, digital signal processors (DSPs), application specific integrated circuits (ASICs), field programmable gate arrays (FPGAs), programmable logic circuitry, or the like, either alone or in any suitable combination.

Sensing circuitry 84 may be configured to switch between one or more sensing vectors via electrodes, such as in an electrode array. In some examples, sensing circuitry 84 may be configured to sense muscle activity, impedance changes and sensing electrical activity of the heart. Though not shown, some examples of medical devices that perform the orientation and activity sensing techniques of this disclosure may include medical devices that are configured to deliver therapy, such as drug therapy, or electrical stimulation therapy. Such medical devices may include therapy generation circuitry instead of or in addition to sensing circuitry, as well as chemical detection, light or temperature detection or other sensors. Some example medical devices may include a cardiac pacemaker, a cardioverter, a defibrillator and a neurostimulator.

In some examples, IMD 10 may additionally or alternatively be configured to sense one or more physiological parameters of patient 2. For example, physiological parameters may include heart rate, electromyography (EMG), an electroencephalogram (EEG), an electrocardiogram (ECG), temperature, respiration rate, or pH. These physiological parameters may be used by processing circuitry 80, in some examples, to confirm or reject changes in sensed orientation and activity state that may result from vibration, patient travel (e.g., in an aircraft, car or train), or some other false positive of orientation and activity state. In some examples, processing circuitry 80 may use heart rate, activity, and/or time of day (as examples) to assume or confirm a patient is upright (awake and active) or laying down (asleep and inactive).

Orientation and activity circuitry 86 allows IMD 10 to sense the patient orientation and activity state, e.g., orientation, activity or any other static orientation or motion of patient 2. Orientation and activity state circuitry 86 may include circuitry suitable for amplifying, filtering or otherwise processing signals received from orientation sensor 87. Orientation and activity state information generated by orientation and activity state circuitry 86 and processing circuitry 80 may correspond to an activity and/or orientation undertaken by patient 2 or a gross level of physical activity, e.g., activity counts based on footfalls or the like.

In the example of FIG. 2, orientation and activity state circuitry 86 may receive signals from orientation sensor 87. Orientation sensor 87 include one or more accelerometers, such as 3-axis accelerometers, capable of detecting static orientation or vectors in three-dimensions. In some examples the one or more accelerometers may be responsive to a gravitational field. For example, orientation sensor 87 may include one or more micro-electro-mechanical accelerometers. In other examples, orientation and activity state circuitry 86 may alternatively or additionally include one or more gyroscopes, pressure transducers or other sensors to sense the orientation and activity of patient 2. Although orientation sensor 87 is described as containing the 3-axis accelerometer, orientation sensor 87 may contain multiple single-axis accelerometers, dual-axis accelerometers, 3-axis accelerometers, or some combination thereof.

In some examples, processing circuitry 80 processes the analog output of the orientation and activity sensor in orientation and activity state circuitry 86 to determine activity and/or orientation data. For example, processing circuitry 80 or a processor of orientation and activity circuitry 86 may process the raw signals provided by the orientation and activity sensor to determine activity counts. In some examples, processing circuitry 80 may process the signals provided by the orientation and activity state sensor to determine velocity of motion information along each axis. In this disclosure, the term “sensor” refers to an orientation and activity state sensor, such as an accelerometer, unless otherwise noted.

In one example, each of the x, y, and z signals provided by the orientation and activity state sensor has both a DC component and an AC component. The DC components may describe the gravitational force exerted upon the sensor and may thereby be used to determine orientation of the sensor within the gravitational field of the earth. In other words, the sensor may be responsive to a gravitational field. Assuming the orientation of the sensor is relatively fixed with respect to patient 2, the DC components of the x, y and z signals may be utilized to determine the patient's body part orientation within the gravitational field, and hence to determine the body orientation of the patient or the patient's body part orientation. In other words, processing circuitry 80 may receive the outputs of the orientation and activity state sensor and determine an orientation vector of the orientation and activity state sensor relative to a gravitational vector of the gravitational field. As a result of determining the orientation of the orientation and activity state sensor relative to the patient's body part orientation, processing circuitry 80 may determine the body part orientation or, in some examples, a posture of the patient. The x, y and z signals may be interpreted by processing circuitry 80 as an orientation vector of the sensor [ax, ay, az].

In some examples, processing circuitry 80 may select one of the reference templates or the for determining the body part angle orientation value based on the orientation vector. Processing circuitry 80 may determine a value representative of the body angle of patient 2 (shown in FIG. 1) based on a dot product of the orientation vector and the selected reference template. In some examples, processing circuitry 80 may not determine the actual angle, for example in degrees, of the body angle. Instead, processing circuitry 80 may use a scaled representation of the dot product of the sensor orientation vector and the reference template to determine a value representative of the body angle, which may simplify the calculations and memory usage for the processing circuitry 80.

In other examples, processing circuitry 80 may calculate a dot product of the orientation vector and each reference template associated with the posture for each of the plurality of postures. Processing circuitry 80 may select the one of the plurality of postures associated with the largest of the of dot products as the current posture of the patient. As described above in relation to FIG. 1, the largest dot product (i.e. large cosine) indicates that angle between the sensor orientation vector and the reference template is small, said another way the angle distance is small and the cosine (value of the dot product is high).

The AC component of the x, y and z signals may yield information about patient motion. In particular, the AC component of a signal may be used to derive a value for an activity describing the patient's motion in one or more combinations of axes. This activity may involve a level, direction of motion, or acceleration of patient 2.

One method for determining the activity is an activity count. An activity count may be used to indicate the activity or activity level of patient 2. For example, a signal processor may sum the magnitudes of the AC portion of an accelerometer signal for N consecutive samples. For instance, assuming sampling occurs as 25 Hz, N may be set to 25, so that count logic provides the sum of the samples that are obtained in one second. This sum may be referred to as an “activity count.”

The number “N” of consecutive samples may be selected by processing circuitry 80 or a processor of orientation and activity state circuitry 86 based on the current orientation and activity state, in some examples. The activity count may be the activity portion of the orientation and activity state parameter value that may be associated with the orientation portion. The resulting orientation and activity state parameter value may then incorporate both activity and orientation to generate an accurate indication of the motion of patient 2. In some examples, the posture portion of the orientation and activity state parameter may include the posture, e.g. upright, lying down, or a combination of back-angle and side-angle. In some examples, the orientation and activity state parameter may include the orientation vector of the sensor.

As another example, the activity portion of the orientation and activity state parameter value may describe a direction of motion. This activity parameter may be associated with a directional activity vector describing activity levels in 3 dimensions. Another example of an activity parameter relates to acceleration in particular directions. A value quantifying the magnitude and orientation of the largest directional change of motion over a period of time may be associated with the activity portion of an orientation and activity state parameter value.

Processing circuitry 80 may store orientation and activity state information in memory 82 for later review by a clinician, used to adjust therapy, present an orientation and activity state indication to patient 2, or some combination thereof. As an example, processing circuitry 80 may record the orientation and activity state parameter value, or output, of the 3-axis accelerometer and assign the orientation and activity state parameter value to a certain predefined posture indicated by the orientation and activity state parameter value. In this manner, IMD 10 may be able to track how often and how long patient 2 remains within a certain posture.

In some examples, processing circuitry 80 may also adjust therapy or sensing (e.g., change an electrode vector, stimulation waveform parameter, or sensing threshold for one or more physiological parameters) for a new orientation when orientation and activity state circuitry 86 indicates that patient 2 has in fact changed postures. Therefore, 1 MB 10 may be configured to provide posture responsive stimulation therapy or sensing to patient 2. Stimulation or sensing adjustments in response to orientation and activity state may be automatic or semi-automatic (e.g., subject to patient approval). In many cases, fully automatic adjustments may be desirable so that IMD 10 may react more quickly to orientation and activity state changes.

An orientation and activity state parameter value from orientation and activity state circuitry 86 that indicates the orientation and activity state may constantly vary throughout the day of patient 2. However, a certain activity (e.g., walking, running, or biking) or a posture (e.g., standing, sitting, or lying down) may include multiple orientation and activity state parameter values from orientation and activity state circuitry 86. Memory 82 may include definitions for each orientation and activity state of patient 2.

Posture responsive stimulation may allow IMD 10 to implement a certain level of automation in therapy adjustments. Automatically adjusting stimulation may free patient 2 from the constant task of manually adjusting therapy each time patient 2 changes posture or starts and stops a certain orientation and activity state. Such manual adjustment of stimulation parameters can be tedious, requiring patient 2 to, for example, depress one or more keys of external device 12 multiple times during the patient orientation and activity state to maintain adequate symptom control. In some examples, patient 2 may eventually be able to receive orientation and activity state responsive stimulation therapy without the need to continue making changes for different postures via external device 12. Instead, patient 2 may transition immediately or over time to fully automatic adjustments based on their orientation and activity state.

Wireless telemetry in IMD 10 with external device 12, or another device may be accomplished, for example, by radio frequency (RF) communication, proximal inductive interaction, or tissue conductance communication (TCC) of IMD 10 with device 12. Telemetry circuit 88 may send information to and receive information from external device 12 on a continuous basis, at periodic intervals, at non-periodic intervals, or upon request from the stimulator or programmer. To support wireless communication, telemetry circuit 88 may include appropriate electronic components, such as amplifiers, filters, mixers, encoders, decoders, and the like.

Power source 90 delivers operating power to the components of IMD 10. Power source 90 may include a small rechargeable or non-rechargeable battery and a power generation circuit to produce the operating power. Recharging may be accomplished through proximal inductive interaction between an external charger and an inductive charging coil within IMD 10. As one example, external device 12 may include the charger to recharge power source 90 of IMD 10. Hence, the programmer and charger may be integrated in the same device. Alternatively, in some cases, a charger unit may serve as an intermediate device that communicates with both the IMD and the programmer. In some examples, power requirements may be small enough to allow IMD 10 to utilize patient motion and implement a kinetic energy-scavenging device to trickle charge a rechargeable battery. In other examples, traditional batteries may be used for a limited period of time. As a further alternative, an external inductive power supply could transcutaneously power IMD 10 when needed or desired.

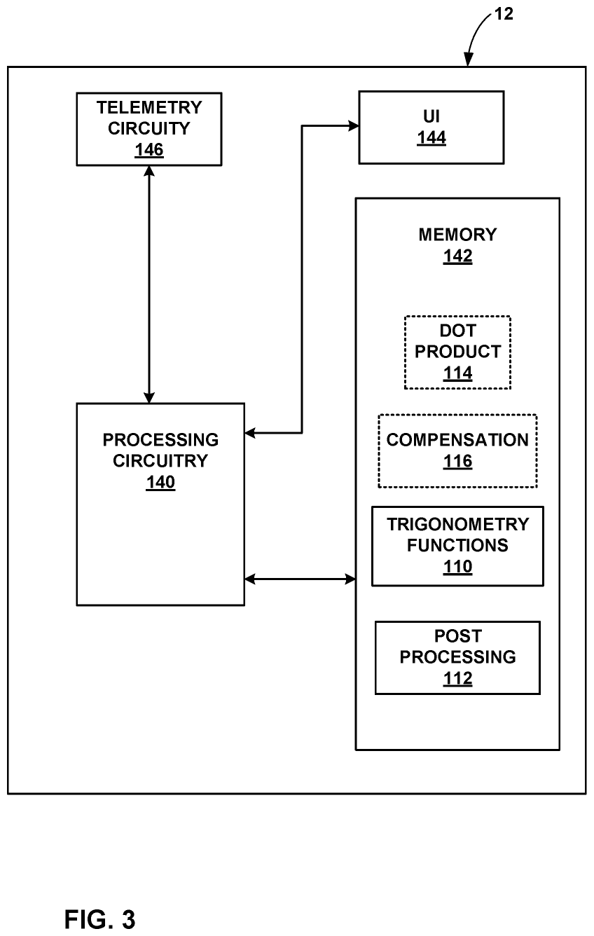

FIG. 3 is a functional block diagram illustrating various components of an external device 12 for IMD 10, as depicted in FIG. 2. As shown in FIG. 3, external device 12 includes processing circuitry 140, memory 142, telemetry circuitry 146, user interface 144, and post-processing circuitry 112. A clinician or patient 2 may interact with user interface 144 to review information received by programmer 12 from IMD 10, such as orientation and activity states over time or other information described herein as being sensed and/or determined by IMD 10, and to program the sensing and/or stimulation functions of IMD 10, e.g., select values for parameters that control such functions.

Memory 142 includes operation instructions for processing circuitry 140 and data related to patient 2 and/or IMD 10, e.g., received from IMD 10. In the example of FIG. 3, memory 142 includes dot product functions 114, compensation functions 116, trigonometry functions 110 and post processing functions 112. Memory 142 may also include operation instructions for other features of external device 12.

As described above, dot product functions 114, also called inner product, is a technique to multiply two vectors and yield the magnitude of the vectors multiplied by the cosine of the angle between the vectors. Dividing the dot product by the product of the vector magnitudes results in a scalar value representing the cosine of the vectors. External device 12, may use dot product functions 114 during compensation, post-processing or other operations.

Compensation functions 116 provide instructions to processing circuitry 140 for determining the orientation of the sensor relative to the patient. In other words, because IMD 10A, depicted in FIG. 1, may be in a relatively fixed position and orientation relative to the chest of patient 2, device orientation compensation allows IMD 10A, or external device 12, to determine the chest orientation of patient 2 by determining the orientation vector of the sensor, based on the sensor outputs. Compensation may also be referred to as calibration in this disclosure, however, calibration also refers to activities during device manufacturing, for example, to account for variability of the sensor's signals, requiring software or hardware trims, or stored sensor calibration factors.

Because the angle measurements may change part of the overall system approach may be to provide a means to update reference measurements to account for movement. For example, for an implanted IMD, the device may shift position until the pocket becomes stable. For a strapped, or otherwise attached device, the device may shift position. In some examples patients may display somewhat consistent lying down positions due to habit as well as substantially self-consistent daily body or body part angle measurements. In other examples given patient's change in sleeping habit, such as from sleeping on a different side, might not cause shifts in the measured orientation angle. In other examples, some patients may have any significant variability in angles due to soft tissue movement. It may be desirable to update references on device-to-patient orientation periodically, either manually or by automated means.

Trigonometry functions 110 may include functions such as cosine, sine, arccosine and similar functions that may be used during compensation, post-processing or other operations. As described above in relation to FIG. 1, processing circuitry 140 of external device 12 may be better suited to more complex calculations, such as trigonometry functions 110 when compared to less complex processing circuitry in IMD 10.

As described above in relation to FIG. 2, processing circuitry 80 of IMD 10 may use simplified calculations for angle measurements, such as a scaled value for the cosine of an angle, rather than calculating the actual angle. In some examples, IMD 10 may transmit physiological information to external device 12, such as posture tracking, sleep position tracking and other information. External device 12 may receive the physiological information in a simplified data form, such as the cosine of an angle, and perform post-processing functions 112 to convert the simplified information into information that may be helpful to the patient and/or a clinician. In some examples, post-processing functions 112 may include converting scalar angles into angles in degrees or radians, creating tables of values, graphs, charts, or other display information.

Telemetry circuitry 146 allows the transfer of data to and from IMD 10. Telemetry circuit 146 may communicate automatically with IMD 10 at a scheduled time or when the telemetry circuit detects the proximity of the stimulator. Alternatively, telemetry circuit 146 may communicate with IMD 10 when signaled by a user through user interface 144. To support wireless communication, telemetry circuit 146 may include appropriate electronic components, such as amplifiers, filters, mixers, encoders, decoders, and the like. In some cases, external device 12 may be used when coupled to an alternating current (AC) outlet, i.e., AC line power, either directly or via an AC/DC adapter.

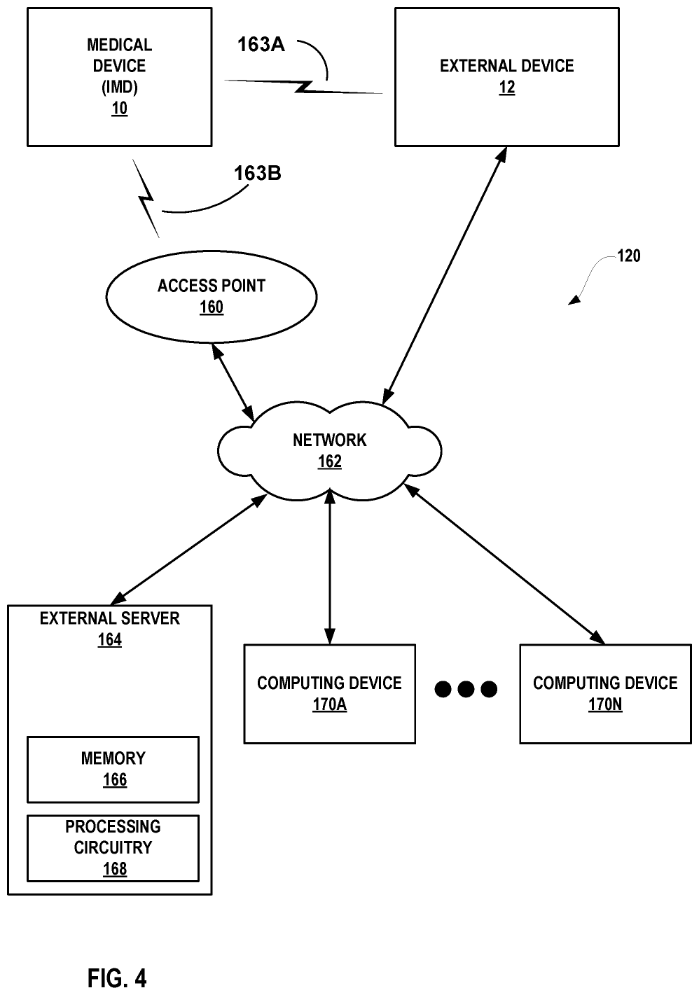

FIG. 4 is a block diagram illustrating an example system 120 that includes an external device, such as a server 164, and one or more computing devices 170A-170N, that are coupled to IMD 10 and external device 12 shown in FIGS. 1 and 2, via a network 162. In this example, IMD 10 may use its telemetry circuit 88 to communicate with external device 12 via a first wireless connection 163A, and to communication with an access point 160 via a second wireless connection 163B.

In the example of FIG. 4, access point 160, external device 12, server 164, and computing devices 170A-170N are interconnected, and able to communicate with each other, through network 162. In some cases, one or more of access point 160, external device 12, server 164, and computing devices 170A-170N may be coupled to network 162 through one or more wireless connections. IMD 10, external device 12, server 164, and computing devices 170A-170N may each comprise one or more processors, such as processing circuitry 168. The one or more processors may include one or more microprocessors, digital signal processors (DSPs), application specific integrated circuits (ASICs), field programmable gate arrays (FPGAs), programmable logic circuitry, or the like, that may perform various functions and operations, such as those described in this disclosure.

Access point 160 may comprise a device, such as a home monitoring device, that connects to network 162 via any of a variety of connections, such as telephone dial-up, digital subscriber line (DSL), or cable modem connections. In other examples, access point 160 may be coupled to network 162 through different forms of connections, including wired or wireless connections.

During operation, IMD 10 may collect and store various forms of data. For example, IMD 10 may collect sensed orientation and activity state information that indicates how patient 2, as depicted in FIG. 1, moves throughout each day. In some cases, IMD 10 may directly analyze the collected data to evaluate the patient 2 orientation and activity state, such as what percentage of time patient 2 was in each identified posture. In other cases, however, IMD 10 may send stored data relating to orientation and activity state information to external device 12 and/or server 164 (e.g., processing circuitry 168 of server 164), either wirelessly or via access point 160 and network 162, for remote processing and analysis, including post-processing functions 112 described above in relation to FIG. 3. For example, IMD 10 may sense, process, trend and evaluate the sensed orientation and activity state information. Alternatively, processing, trending and evaluation functions may be distributed to other devices such as external device 12 or server 164 (e.g., processing circuitry 168 of server 164), which are coupled to network 162. In addition, orientation and activity state information may be archived by any of such devices, e.g., for later retrieval and analysis by a clinician. For example, server 164 may archive orientation and activity state information, which may include patient body angle, at memory 166.

In some cases, external device 12 or server 164 may process orientation and activity state information, raw orientation data, other physiological sensing information, and/or therapy information into a displayable orientation and activity state report, which may be displayed via external device 12 or one of computing devices 170A-170N. The orientation and activity state report may contain trend data for evaluation by a clinician, e.g., by visual inspection of graphic data. In some cases, the orientation and activity state report may include the number of activities patient 2 conducted, a percentage of time patient 2 was in each orientation and activity state, the average time patient 2 was continuously within an orientation and activity state, and information regarding sensing and therapy delivery during each activity, or any other information relevant to patient 2 therapy, based on analysis and evaluation performed automatically by IMD 10, external device 12 or server 164. A clinician or other trained professional may review and/or annotate the orientation and activity state report, and possibly identify any problems or issues with the physiological sensing or therapy that should be addressed.

In some cases, server 164 may be configured to provide a secure storage site for archival of orientation and activity state information that has been collected from IMD 10 and/or external device 12. Network 162 may comprise a local area network, wide area network, or global network, such as the Internet. In some cases, external device 12 or server 164 may assemble orientation and activity state information in web pages or other documents for viewing by trained professionals, such as clinicians, via viewing terminals associated with computing devices 170A-170N. System 120 may be implemented, in some aspects, with general network technology and functionality similar to that provided by the Medtronic CareLink® Network developed by Medtronic, Inc., of Minneapolis, Minn.

Although some examples of the disclosure may involve orientation and activity state information and data, system 120 may be employed to distribute any information relating to the treatment of patient 2 and the operation of any device associated therewith. For example, system 120 may allow therapy errors or device errors to be immediately reported to the clinician. In addition, system 120 may allow the clinician to remotely intervene and reprogram IMD 10, external device 12, or communicate with patient 2. In an additional example, the clinician may utilize system 120 to monitor multiple patients and share data with other clinicians in an effort to coordinate rapid evolution of effective treatment of patients.

Furthermore, techniques of this disclosure may be applicable to IMDs that convey other therapies in which orientation and activity state information is important, such as, e.g., deep brain stimulation (DBS), pelvic floor stimulation, gastric stimulation, occipital stimulation, functional electrical stimulation, cardiac pacing, anti-tachycardia therapies, and the like. Also, in some aspects, techniques for evaluating orientation and activity state information, as described in this disclosure, may be applied to IMDs that are generally dedicated to sensing or monitoring and do not include stimulation or other therapy components, such as IMD 10.

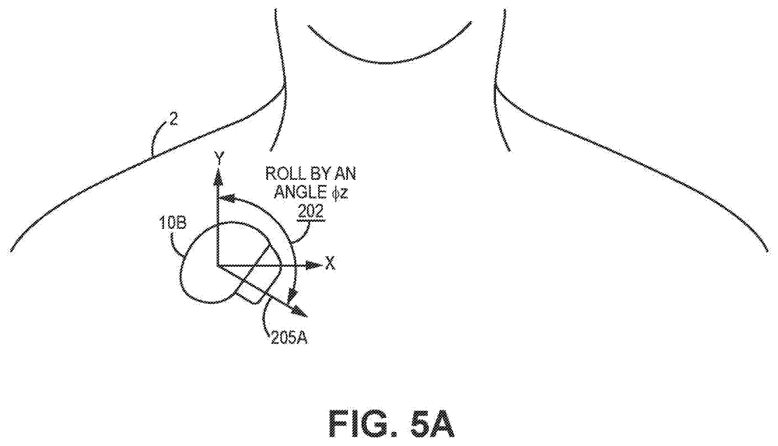

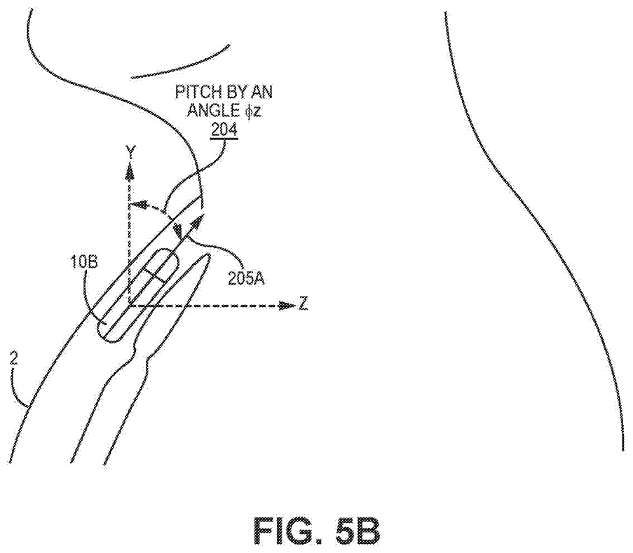

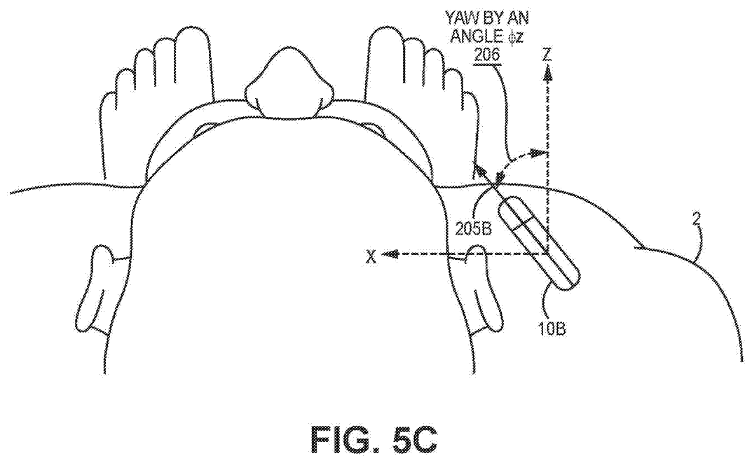

FIGS. 5A-5C are conceptual diagrams illustrating the roll, pitch and yaw angles of an example medical device which may be worn by or implanted in a patient. The techniques illustrated by FIGS. 5A-5C may apply to either a medical device attached to a patient, e.g. with an arrangement of straps, or implanted in a substantially fixed orientation and orientation, e.g. in a tight pocket and, in some examples, sutured in place to minimize movement.

3-Axis accelerometers have been used externally for short periods of time and placed on or about the body surface to monitor the body orientation/position, activity level and/or orientation and/or movement of specific limbs, or sections of the body for various purposes such as to aid in the diagnosis of various movement disorders, or automatically detecting falls. 3-axis accelerometers may also be used in implanted devices to do the same types of monitoring, but for a much longer period of time, e.g. months to years. Medical devices that include such 3-axis accelerometers may be useful to measure activity and body position and orientation parameters that can help recognize when patients are getting sicker, such as those suffering from heart failure, also called congestive heart failure (CHF). Such parameters may be combined and complemented with other physiologic metrics that also relate to the health condition of a patient, such as activity, impedance, body temperature, blood pressure, and heart rate.

Some proposed daily metrics that can be extracted from a 3-axis accelerometer may include:
Amount of time upright
Amount of time upright and inactive (likely sitting)
Amount of time upright and active (likely walking, although other activities may be possible)
Amount of time active (where patient may be active when not upright)
Amount of time laying in any position and/or orientation
Average laying angle
Average overall body angle
Sleep quality, which may be determined based, at least in part, on a number of turns or other position/orientation changes at night, and/or a number of major interruptions at night (e.g., bathroom breaks)
Fall detection, e.g., number of falls
Detection of patient tapping the device, e.g., to provide user input, also referred to as “taptivation.”
   In some examples, a medical device may use the orientation and activity state of a patient to direct the use of other sensors (e.g. via TCC) that may show posture dependency, such as pressure sensors or sensors that detect patient fluid status, such as via thoracic or tissue impedance. In some examples, a medical device may use the orientation and activity state to recognize that a patient has been in a given body position long enough for physiological parameters, such as fluid levels, to reach a stable and repeatable equilibrium.

To correctly monitor body or body part orientation the axes of the accelerometers should be correctly aligned with the body or body part, or implement compensation means to correct for misalignment. In most external applications, the 3-axis accelerometers can be aligned in a consistent manner from subject to subject, by using holsters on a belt, or chest straps or tape. Device orientation compensation, however may still be necessary to account for differences in body shapes or differences in the orientation of body parts from patient to patient. The degree of compensation depending on the required accuracy of the orientation measurements. The longer timeframe of implanted devices means implanted devices may be subject to changes in their orientation with time, for example from a patient's twiddling with the devices, soft tissue effects or other device migration. The device orientation compensation settings, as described in this disclosure, may be updated periodically to account for these changes. For example, a recompensation may be accomplished during a normal patient follow up visit with a clinician.

When utilizing a 3-axis accelerometer or other orientation sensor in an implanted device, not only do differences in body shape and orientation of body parts cause variability of the implant orientation of the internal 3-axis accelerometer relative to the earth's gravity, but the orientation of the implant of the device may be chosen to, for example, for best use of the main diagnostic or therapeutic function of the device. For example, a medical device may be implanted in a position and orientation chosen to best receive ECG signals. Other factors in implanted device orientation and position may include to position the scar of the implant to minimize the visibility of the scar and optimize the performance of other sensors onboard the device, e.g. pH monitor, for example.

FIG. 5A illustrates an example roll angle 202 of the rotation of IMD 10B, with orientation vector 205A of a sensor, such as a 3-axis accelerometer. IMD 10B may include features similar to IMD 10A and IMD 10 as described above in relation to FIGS. 1 and 2. In the example of FIG. 5A, roll 202 is the angle around the ideal front-to-back central axis of the device and the angle of rotation is measured relative to the long axis of the patient. The long axis is a line running through the spine from the crown of the patient's head through the heels and is parallel to the Y-axis depicted in FIG. 5A. In other words, the orientation vector 205A is rotated at the roll angle 202 relative to the long axis of patient 2. IMD 10B may be substantially similar to IMD 10, or may be a different IMD, e.g., a cardiac pacemaker, cardioverter, and or defibrillator. IMD 10B may include circuitry, such processing circuitry 80, memory 82, orientation sensor 87, and orientation and activity state circuitry 86, described above with respect to IMD 10 in FIG. 2. IMDs 10, 10A and 10B may implement any of the techniques described herein with respect to any similar medical device. The implantation locations of IMDs 10 and 200 illustrated herein are merely examples, and medical devices implanted anywhere within patient or attached to the patient, e.g. with a harness or straps, may implement any of the techniques described herein.

FIG. 5B illustrates an example pitch angle 204 of IMD 10B as implanted in the patient. In the example of FIG. 5B, pitch 204 is the rotation angle about the left-right axis of the patient/device and is measured from a line parallel to the spine of patient 2. Orientation vector 205A is rotated about the left-right axis at the pitch angle 204.

For some applications, it may be necessary to account for the yaw of the implant. FIG. 5C illustrates an example a yaw 206 of device 200. In FIG. 5C, yaw is the rotation angle about the long axis of the patient, which is the Y-axis pointing straight out of FIG. 5C. Roll 202 is rotation measured from the long axis as compared to yaw 206, which is rotation about the long axis. Orientation vector 205A is used in FIG. 5C for clarity and is pointing 180 degrees from orientation vector 205A as shown in FIGS. 5A and 5B. Determining and compensating for yaw 206 may be valuable when it is important to recognize what position the patient is in when they are lying down. Laying down positions include supine (face up) and prone (face down), along with other position such as right shoulder up, and so on.

The example axis convention in this disclosure is just one example of possible conventions. The techniques of this disclosure apply equally to other conventions as shown in the table below.

| | Superior/ | | | | |
|---|---|---|---|---|---|
| | Inferior/ | Lateral/ | Anterior/ | Polarity | |
| Axis | (top/bottom | Medial | Posterior | to Gravity | RH or LH |
| Conventions | or long axis) | (right/left) | (front/back) | Convention | Coordinates? |
| Selected | Y + Superior | X + Medial | Z + Anterior | +Up | RH |
| convention | | | | | |
| in this | | | | | |
| disclosure | | | | | |
| Example 1 | X + Inferior | Y + Medial | Z + Anterior | +Down | RH |
| Example 2 | Y + Superior | X + Lateral | Z + Anterior | +Up | LH |
| Example 3 | Y + Inferior | X + Medial | Z + Posterior | +Down | RH |

For clarity, the conventions listed in the first row of the above table will be followed for Phix (ϕx), Phiy (ϕy), and Phiz (ϕz). In other words, all rotations are in a right-hand manner and the axes follow the right-hand rule. For example, Phix rotates in a right-hand way about the X-axis and is 0 degrees at the +Y axis, 90 degrees at the +Z axis on the XZ plane, and 180 degrees at the −Y axis. The other two axes follow this same convention, where the rotation Phiy rotates about the y-axis, and starts at 0 on the +X axis, to 90 degrees at the +Z axis, to 180 degrees on the −X axis. Phiz rotates about the z axis and is 0 degrees at the +X axis.

Converting the convention in row 1 for an accelerometer that follows the convention of example 2 may be done by inverting the x-axis output, or alternately switching the x and y axis definitions, and reorienting the accelerometer to have the new y axis pointing up. In another example, an accelerometer may be used with the convention as listed in example 3 by inverting the y and z axes so y and z point up when positive.

Using the conventions described above, this disclosure describes techniques for a medical device processor, such as processing circuitry 80 depicted in FIG. 2, to determine orientation information by receiving outputs of a sensor that is responsive to a gravitational field, such as a 3-axis accelerometer. Based on the outputs of the sensor, a processor may determine an orientation vector of the sensor relative to a gravitational vector of the gravitational field. Also, by determining the orientation of the sensor in the medical device relative to the patient, i.e. determining the roll, pitch and yaw of the sensor relative to the patient, the processor may determine posture information of patient 2. In this disclosure, the process of determining the orientation of the sensor relative to the patient is called device orientation compensation, or simply compensation. In some examples the patient may be placed in a predetermined compensation orientation posture, e.g. upright.

As described above in relation to FIG. 2, processing circuitry 80 in IMD 10 may be selected as a low power, and less complex processor than the processor in external device 12. A lower power, simpler processor may have advantages in reduced operating power consumption and therefore in a longer battery life for power source 90. Additionally, the techniques of this disclosure simplify the calculations for determining the orientation of patient 2. In some examples, processing circuitry 140 in external device 12, depicted in FIGS. 1 and 3, may include a higher power, more complex processor. More complex mathematical functions, such as trigonometry functions such as arccosine, arcsine and similar functions may be more efficiently handled by external device 12 rather than IMD 10. Therefore, for the compensation process, IMD 10 may transmit the sensor outputs to external device 12. External device 12 may perform the compensation calculations, which may involve more complex trigonometry and other functions. External device 12 may then transmit the compensation information back to IMD 10. IMD 10 may then use the compensation information to perform simplified calculations to determine patient orientation.

In some examples, IMD 10 may transmit to an external computing device, such as external device 12, the orientation vector of the 3-axis accelerometer, based on the outputs of the 3-axis accelerometer, wherein the patient is in a predetermined compensation orientation, such as an upright orientation. External device 12 may perform compensation calculations and transmit one or more reference templates to IMD 10. IMD 10 may store the reference templates at a memory location, such as memory 82, which is operatively coupled to the processing circuitry 80. Processing circuitry 80 may perform simplified calculations to determine patient orientation using the reference templates. Periodically, the reference templates may be updated in memory 82 of IMD 10 to account for any movement of IMD 10 relative to patient 2, for example, during a follow up visit at a clinician. In some examples, IMD 10 may include an algorithm to automatically verify and compensate for some migration of IMD 10 after implant. In some examples the automatic compensation algorithm may compensate for only one of the angles, such as roll 202. In other examples, the automatic algorithm may compensate for two or more of the angles.

One example of a device orientation compensation procedure may begin with the patient in a predetermined posture, such as upright. In some examples, activity, heart rate, time of day, or the like may be used by processing circuitry 80 to determine whether patient is upright. Additionally or alternatively, processing circuitry 80 may sense tapping of the device by the patient or clinician indicating the patient is upright or in another calibration posture via the accelerometer.

The system, such as system 1 depicted in FIG. 1, may gather the data from all three axes of the sensor. In some examples, the three sensor outputs may be filtered using a 0.5 second average, or one-half Hertz (½ Hz) filter or other similar low frequency filter such as 1 Hz. One single measurement of the ax, ay, and az sensor values for this posture may then be used for rotation angle calculations. The orientation vector [ax, ay, az] of the 3-axis accelerometer is used to determine the compensated rotation vector [ax′, ay′, az′], which may be used to determine reference templates.

Equations (1)-(14) below demonstrate one way to calculate roll and pitch using the upright posture as a predetermined compensation reference posture assuming no yaw:

Phix=arcsin(−az′) (1)

Phiz=arcos(ay′/cos(Phix)), (for Phix≠90°) (2)

Equations (1) and (2) result in four pairs of solutions:

Phix1=arcsin(az′), (3)

and if 0≤Phix1≤180 the second solution for Phix is:

Phix2=180−Phix1, (4)

but if −180≤Phix1≤0 the second solution for Phix is:

Phix2=−180−Phix1. (5)

The two solutions above are then associated with the four Phiz solutions via equation (2):

Phiz 1 and Phiz 2=±arcos(ay′/cos(Phix1) (6)

Phiz 3 and Phiz 4=±arcos(ay′/cos(Phix2) (7)

The relationships above may be summarized as shown in the table below. Using the ax′ and ay′ sign values and the table above one can identify which of the above 4 Phiz values is within the desired range, the correct Phix value is the one associated with the correct Phiz value.

| ax′ | ay′ | Phiz Range |
|---|---|---|
| + | + | 0° < Phiz < 90° |
| 0 | + | Phiz = 0° |
| + | 0 | Phiz = 90° |
| 0 | 0 | Phiz = NA |
| − | − | −180° < Phiz < −90° |
| 0 | − | Phiz = either ±180° |
| + | − | 90° < 0 < 180° |
| − | + | −90° < Phiz < 0° |
| − | 0 | Phiz = −90° |

To identify the correct Phiz values use magnitude and the sign values:

Sign(Phiz)=Sign(ax′) for ax≠0. (8)

If (ay′>0 and ax≠0) then choose Phiz such that 0<|Phiz|<90° (9)

If (ay′<0 and ax≠0) then choose Phiz such that 90<|Phiz|<180° (10)

There are specific ax′ and ay′ boundary values that result in values of Phiz with a simplified calculation. Some boundary values may not necessarily be correctly defined using equations (1) and (2):

If (ax′=0 and ay′>0) then use Phiz=0 (11)

If (ax′=0 and ay′<0) then use Phiz=±180 (12)

If (ay′=0 and ax′>0, then use |Phiz|=90° (13)

If (ay′=0 and ax′=0, then use Phiz=Not Available or NA (14)

Using equations (1)-(11), external device 12, as depicted in FIGS. 1 and 3, may estimate the pitch of the implant Phix, and the roll of the implant, Phiz, from the ax, ay, and az values calculated in the upright posture. In some applications, it may be useful to identify the postures when the patient is lying down when using the example equations (1)-(11) above to determine pitch 204 and roll 202. For example, it may be useful to identify when the patient is laying on their back (supine), or to determine what percentage of time they are sleeping in a given posture. It may also be useful to determine the angle at which they are sleeping. In applications to determine lying positions, it may be desirable for IMD 10 to account for the yaw angle of the implant. In a fall detection algorithm, it may not be important to know the orientation of the patient when they are on the ground, therefore a yaw correction may unnecessary, and the next yaw calculation section can be skipped with a value of zero degrees (0°) substituted for the yaw value.

One example technique to determine yaw angle 206 of the sensor, e.g. a 3-axis accelerometer which may be included in orientation sensor 87, as depicted in FIG. 2, may begin with placing the patient in a predetermined posture. Some examples may include a lying down posture such as face up (supine), face down (prone), left-side up, or right-side up. The system, e.g., processing circuitry and memory of one or more devices of system 120 shown in FIG. 4, such as IMD 10 or external device 12, may then store which second predetermined posture was used. Similar to described above for determining roll 202 and pitch 204 from the upright posture, system 1, depicted in FIG. 1, may gather the data from all three axes of the sensor, for example using a 0.5 second average, or ½ Hz filter. The three ax, ay, and az values for the selected lying posture, should then be stored for the example yaw angle calculations, described below.

In summary, the example technique to calculate yaw calculates the roll and pitch per the approach described above using equations (1)-(11) from the upright posture. Then compensate the second set of data from the second posture to remove the effects of the measured roll and pitch rotation using the following formula:

(axcorr,aycorr,azcorr)=Rx−1*Rz−1*(ax,ay,az). (15)

where: Rz−1 (inverse roll), and Rx−1 (inverse pitch) are 3×3 matrices:

Rz-1 (inverse roll)

| | | |
|---|---|---|
| cos(Phiz) | −sin(Phiz) | 0.00 |
| sin(Phiz) | cos(Phiz) | 0.00 |
| 0 | 0 | 1 |

Rx-1(inverse pitch)

| | | |
|---|---|---|
| 1 | 0 | 0 |
| 0.00 | cos(Phix) | −sin(Phix) |
| 0.00 | sin(Phix) | cos(Phix) |

In the Rz−1 and Rx−1 matrices, Phiz and Phix are the previously estimated roll and pitch correction. According to this example, after correction for roll and pitch angle, for each chosen posture, there are two equations each that can be used to calculate the yaw. For an accurate representation for Phiy it may be desirable to use arcsine for small angle outputs, e.g., less than ±45 degrees, and arccosine for larger angles, e.g., greater than 45 degrees. Therefore, in some examples, processing circuitry may apply the following rule to calculate the yaw values in the equations below: if |axcorr| or |azcorr|>0.707 (from equation (15) above), use the arcsine function, otherwise use the arccosine function for the equations.

Supine Yaw Equations:

Phiy=arcos(azcorr) or (16)

Phiy=arcsin(−axcorr) (17)

Face Down Yaw Equations:

Phiy=arcos(azcorr) or (18)

Phiy=arcsin(axcorr) (19)

Left Side Up Yaw Equations:

Phiy=arcos(axcorr) or (20)

Phiy=arcsin(azcorr) (21)

Right Side Up Yaw Equations:

Phiy=arcos(−axcorr) or (22)

Phiy=arcsin(azcorr) (23)

During calibration processing circuitry may compute reference orientation templates with the calculated yaw 206, pitch 204, and roll 202 rotation angles. During the operation, by comparing the outputs of the sensor (ax, ay, az) to five orthogonal posture vectors, IMD 10 may determine the posture of the patient is closest to one of the five ideal basis postures. Five examples of ideal basis posture vectors where the scales are adjusted so that 0=0 G and 1=1 G, may include:

Upright: (0,1,0)

Face Up: (0,0,1)

Face Down: (0,0,−1)

Right-Side Up: (−1,0,0)

Left-Side Up: (1,0,0)

The five orientation vectors above give a basic description of coarse patient orientations. In other examples, a more detailed estimate of patient orientation may include ten reference templates, such as upright, sitting forward, sitting back, sitting left side, sitting right side, lying left side, lying right side, lying face down, lying face up, and upside down. In other words, IMD 10 may store ten reference templates and compare the outputs of the sensor to the ten reference templates to determine which posture of the ten reference templates the patient is closest to their actual posture. In other examples, IMD 10 may store other reference templates used to determine patient body angle, or to compare dot products to threshold ranges to determine patient posture.

These basis posture vectors must be rotated (i.e. compensated for device orientation) using the estimated yaw 206, pitch 204, and roll 202 values to compute the five [(ax′, ay′, az′)] reference templates expected for the device orientation. The actual device orientation values are calculated using equation (24) below and the defined Rx, Ry, and Rz rotation matrices below. Note that these basis posture vectors also have to be multiplied by a device scale factor to match the expected ranges of the values stored in the particular device, otherwise, they do not represent true values as recorded in the device. The reference templates may then be transmitted by external device 12 and stored in a memory location of IMD 10 along with the original measured vectors used for compensation (e.g. upright vector). In other examples, the estimated yaw 206, pitch 204 and roll 202 angles may also be stored in a memory location either of IMD 10, or in another memory location such as computing devices 170 shown in FIG. 4. During post-processing, external device 12, for example may use the patient posture determinations from IMD 10 and the measured vector used for compensation to convert the orientation values from IMD 10 into angle measurements in degrees or radians.

IMD 10 may determine the patient posture, or in other examples the patient's body angle, based on the reference templates. IMD 10 may output the reference posture compensation measurement (that is used to estimate yaw 206, pitch 204 and roll 202 angles), along with time stamped posture information to external device 12, or via access point 160 depicted in FIG. 4, for post-processing of the patient's sleep postures, or other orientation processing by external device 12, server 164 or other computing devices 170. To simplify the discussion, the description below may refer to implanted devices, however the description applies equally to any device fixed in a known orientation on a patient.

The following rotation matrices can be used to calculate the effects of implant pitch, yaw and rotation on a measured value of the accelerometer signals (ax, ay, and az) from a 3-axis accelerometer. When compared to the inverse roll matrix (Rz−1), described above, the rotation matrix for roll (Rz) has sin (Phiz) and negative sin(Phiz) transposed.

Rz (roll)

| | | |
|---|---|---|
| cos(Phiz) | sin(Phiz) | 0.00 |
| −sin(Phiz) | cos(Phiz) | 0.00 |
| 0 | 0 | 1 |

Ry (Yaw)

| | | |
|---|---|---|
| cos(Phiy) | 0.00 | −sin(Phiy) |
| 0 | 1 | 0 |
| sin(Phiy) | 0.00 | cos(Phiy) |

Rx (Pitch)

| | | |
|---|---|---|
| 1 | 0 | 0 |
| 0.00 | cos(Phix) | sin(Phix) |
| 0.00 | −sin(Phix) | cos(Phix) |

[ax′,ay′,az′]=Rz*Rx*Ry*[ax,ay,az] (24)

For the upright reference template, substitute the upright vector (0,1,0) into equation (24) i.e. [ax, ay, az]=[0,1,0]. Multiplying out the matrices:

ax′=sin(Phiz)cos(Phix) (25)

ay′=cos(Phiz)cos(Phix) (26)

az′=−sin(Phix) (27)

The remaining reference templates may be determined in a similar manner. In the example in which IMD 10 determines a patient body angle, IMD 10 may store only the upright reference template, the supine (face-up) reference template and the right-side up reference template. In other examples, the system may use other vectors for compensation, e.g. left side up, which may depend on patient capability or other factors.

External device 12 depicted in FIGS. 1 and 3, may determine the pitch angle, Phix from equation (27) as:

Phix=arcsin(−az′) (28)

The rotation angle Phiz can then be calculated from either equation (25) or equation (26), or both. Selecting equation (26):

Phiz=Arcos(ay′/cos(Phix)). (29)

In some examples, external device 12 may determine the remaining reference templates from the single, predetermined upright reference posture, based on equations (25)-(29). In other words, processing circuitry 140 of external device 12, depicted in FIG. 3, may determine all the remaining reference templates based on receiving sensor information from IMD 10 for orientation vector 205A while patient 2 is in the upright posture. That is, the upright posture is the only predetermined reference compensation posture, and the results of compensation in the upright posture allows external device 12 to determine any remaining reference templates by geometrical transformations. Equations (28) and (29) may also be used during post-processing.

The techniques of this disclosure may have advantages over other techniques of determining reference templates and posture or orientation. Selecting the starting posture as upright [0,1,0], and selecting yaw as the first rotation angle, the yaw value, Phiy, has no effect on the measured values of ax′, ay′ and az′. This is only the case for this upright posture, or if the patient were standing on their head. In other words, for the coordinate system convention of this example, selecting the upright posture as the predetermined patient reference posture for compensation, and selecting the order of calculation for the rotation matrices as yaw, pitch then roll, simplifies the calculation of the rotation matrices. For example, choosing the order of angle rotations so that the roll angle is last, can create a direct relationship between the first two angles of rotation of the sensor in the medical device. Two rotation angles may define the plane of the body surface at the site of implant or attachment.

Also, by choosing yaw to be first and pitch to be second, the measurement of pitch can be made with the upright posture of the patient, because in the upright posture any arbitrary yaw value has no effect on the upright measurements of ax, ay and az, and the calculations to measure pitch and roll can then be easily made independently of the yaw. Choosing an upright posture as the reference compensation posture may be a convenient posture for patient 2. For example, an elderly patient may find it more difficult to place themselves in some postures and to move between a variety of postures. The techniques of this disclosure allow for a single reference compensation posture, such as an upright posture.

The procedure of selecting yaw, then pitch then roll corresponds to multiplying the rotation matrices in sequence per equation (24). For example, assume that initially, patient 2 is lying flat on their back on the procedure table, and that the orientation vector for the sensor is aligned with orientation vector's long y-axis parallel to the spine, and sensor vector's x-axis parallel to an arm-to-arm cross-chest line. For the selected coordinate convention, this corresponds to a completely un-rotated device, with the axes of the sensor in IMD 10 perfectly aligned with the patient axes.

The examples shown in FIGS. 5A-5C and described below refer to IMD 10B, which is implanted in the chest of patient 2. In other examples, such as IMD 10 as shown in FIG. 1, the device may be implanted or strapped in other locations and the process may be modified as needed for the selected location. Selecting yaw 206 as the first angle of rotation, which for the selected coordinate convention of this disclosure, is defined as the rotation angle around the long y-axis of IMD 10B, as depicted in FIG. 5C. Yaw 206 is used to align the device so that that the x-axis of the device is parallel to the surface of the skin at the implant site. In the illustrated example, the y-axis is still parallel to the spine and the plane of IMD 10B is not yet parallel to the plane of the implant. This orientation may be referred to as the “yawed IMD” orientation.

In some examples, pitch 204 is selected as the second angle of rotation, as depicted in FIG. 5B. The pitch angle corresponds to the angle of rotation around the x-axis of the IMD 10B to have the orientation of the x-y plane of the “yawed IMD” such that it is perfectly parallel to the surface of the skin at the center of the implant site. During this rotation, the x-axis of the yawed IMD remains parallel to the plane of the waist of the patient, and if the patient were standing upright the x-axis of the yawed and “pitched IMD” orientation is still parallel to the ground.

By defining the yaw angle followed by defining the pitch angle 204, the plane of the implant site, based on the direction of orientation vector 205A, has now been defined by these two rotations. The implant plane is parallel to orientation vector 205A and the x-axis of the implant plane is still parallel to the waist of the patient, as depicted in FIGS. 5A and 5C. The third and final angle of rotation, roll, corresponds to the rotation about the central anterior/posterior z-axis of the device in place at the center of the implant site, as depicted in FIG. 5A. The yaw, pitch, roll sequence allows for a simplified compensation of the reference template for the upright vector [0,1,0], as described above for equations (25)-(27). In some example, external device 12 may calculate any remaining reference templates based on the calculations for the upright reference template.

FIG. 6 is a conceptual diagram illustrating an example technique to determine a patient's chest orientation by determining a back-angle and a side-angle. In some examples, the two orientation angles alone may be used to characterize the daily body positions of patient 2 and, in some examples, used to recognize when and how they are oriented when sleeping. Back-angle 310 is the angle of the chest relative to the ground. When the patient is standing upright, back-angle 310 is in the same direction as the upright reference vector 302. In other words, back-angle 310 is the angle, relative to the ground, of a vector 302 running from heels through the crown of the head of patient 2. Side-angle (not shown in FIG. 6) is the angle such that when the patient is upright, it describes the tilt of the patient left and right, and when laying down, it describes the rotation of the patient's body around their long axis, describing the angle they are facing when in bed. Side-angle allows distinguishing left side up from right side up from lying on the back (supine) to lying face down (prone). Side-angle is the angle of the right-side reference vector 312 running from the left shoulder to the right shoulder. The right-side reference vector 312 is parallel, and in the opposite direction, to the X-axis as described above in relation to FIGS. 5A-5C. In other examples, the side-angle may be determined using a left-side reference vector (not shown in FIG. 6).

An advantage of the techniques of this disclosure include using similar calculation for both upright and laying down vectors. Also, that that the two-dimensional plots of patient chest position during post-processing may be easy to visualize and more intuitive to translate when compared to other approaches. In other words, the techniques of this disclosure may allow plot outputs that are easy to interpret for both a caregiver and the patient and may allow better patient care.

Determining the back-angle 310 and side-angle, defines the patient body angle. When referring to a specific body part, the body angle may be referred to as the body part angle. The patient body angle may coincide with a posture as described above in FIGS. 1-5C. For example, when back-angle 310 is parallel to the gravity vector 300, patient 2 is in an upright posture. In another example, when back-angle 310 is orthogonal to gravity vector 300 (or near zero) and right-side reference vector 312 is as shown in FIG. 6 with the vector pointing out of the page, patient 2 is in a supine posture. When back-angle 310 is orthogonal to gravity vector 300 and right-side reference vector 312 is in the opposite direction as shown in FIG. 6 with the vector pointing into of the page, patient 2 is in a face-down, or prone, posture. The value of the back-angle may be considered indicative of the body part angle relative to the central axis of the body part relative to earth's gravity vector. The value of the side-angle may be considered a body part angle indicative of an angle describing the body part orientation in terms of the body parts rotation about an axis of the body part

Said another way, the two position approach may be used to determine which category of positions the patient's body part may be oriented by determining threshold ranges for the back-angle and side-angle. In the example of a device implanted in a patient's chest, processing circuitry may determine the patient's posture by determining categories of positions based on combinations of ranges for the back-angle and the side-angle. For example, when back-angle 310 is between orthogonal and 45 to the gravity vector, while the side-angle is as shown in FIG. 6, the patient's orientation may be categorized as a face-up, reclined posture. By defining combinations of ranges for the back-angle and the side-angle, any number of categories of positions may be defined. In some examples, such as patients with a congestive heart condition may relieve discomfort by resting in a “relief position” which can be categorized as seated, tilted forward with elbows on knees. A system that includes the two position approach may be able to track how often and how long a patient is in certain categories of positions, which may help caregivers derive activities of daily living and assist the caregiver to diagnose and treat the patient's conditions.

Some advantages of defining the patient body angle with the two position angle approach include providing a range of body angles without having to store one reference template for each body angle of interest. Said another way, the two position angle approach may allow a more granular and less coarse determination of the daily body positions of patient 2. Compared to the reference template approach, the two position angle approach does not require a distance calculation (e.g. a dot product) for each different body angle reference template. As with the reference template approach, the angle calculations do not require a processor intensive arccosine function calculation or look up table. In this example approach, a linear fit to the arccosine curve simplifies the angle calculations, so that the device processor can do the calculation more efficiently, e.g. the processing circuitry of IMD 10 or external device 12 depicted in FIG. 1.

The two position approach may still use the one-posture compensation for finding the device orientation described above in relation to FIGS. 5A-5C. Similar to the reference template approach, an external device with a more complex processor may do the complicated transformative mathematics to generate reference templates, then download the posture reference templates to the medical device. However, the two position angle approach downloads and stores only three specific references templates. With the three orthogonal reference templates, the medical device may determine a unique two body angle description of the patient's position at any point in time with the simplified calculation approach.

Processing circuitry implementing the two position angle approach may switch between orthogonal references, which may make the angle calculation more accurate for a low-resolution accelerometer data (e.g. an 8 or 9 bit accelerometer). This body position can be graphed in a 2D chart during post-processing and may be used to track the body angles in an intuitive manner for review directly as a 2D angle chart, or as a pattern of positions that can be characterized and compared for changes in patient health status. During post-processing, an external device may determine postures by comparing the body angle positions to angle thresholds. For example, by comparing the body angles to thresholds processing circuitry in an external device may determine which body angles are in the orthogonal primary postures of upright, lying face down, lying face up, lying on left side, lying on right side, or any sleep angles of interest.

The three reference templates include upright reference vector 302, supine, or laying reference vector 304 and right-side up reference vector 312. The three reference templates are each orthogonal to each other. As described above in relation to FIGS. 5A-5C, an external device, e.g. external device 12 depicted in FIGS. 1 and 3, receives device orientation vector information from the medical device from an initial measurement of the accelerometer values with the patient in an upright posture. The external device determines the orientation of the device orientation vector in relation to gravity vector 300. External device 12 then uses rotational matrix calculations to create the three orthogonal posture reference values that are loaded into the medical device. In other words, the external device uses the device yaw, pitch and roll values to calculate the supine reference template 304 and right-side-up reference template 312 used later for the body angle calculations.

During operation, processing circuitry, e.g. within the medical device, calculates the two angles (back-angle 310 and the side-angle) using the less computationally intensive dot product calculations. In some examples, processing circuitry may perform calculations with scaled 8-bit numbers. To avoid having to do relatively more complicated arc-cosine calculations, this two posture angle approach uses the three orthogonal references, switching between references at a threshold angle transitions to maintain a near linear relationship between the accelerometer output and the body angle. In some examples, the threshold angle is approximately 45 degrees. In some examples, to determine whether to switch between references, processing circuitry may determine the angle to the supine reference template 308 and the angle to the upright reference template 306.

For calculations by the medical device, the 8-bit numbers may be stored as re-scaled values. In some examples, post-processing, as described above in relation to FIG. 3, may be used to scale the numbers back up to values (e.g. degrees or radians) that may be used by a clinician. In other examples, the external device 12 may receive the accelerometer sensor values and directly calculate the patient body angle in degree values rather than unscaled values.

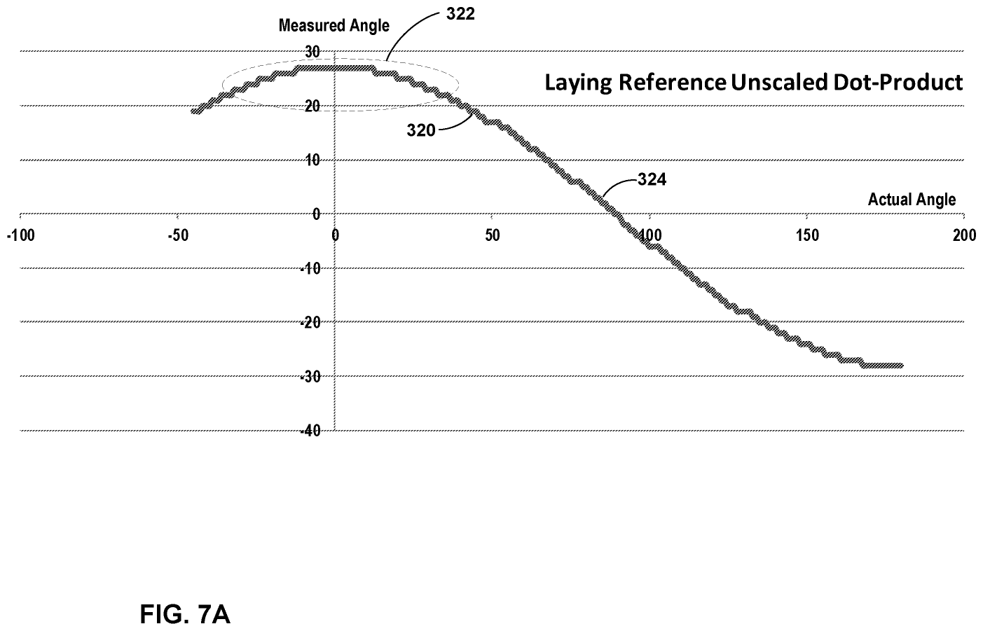

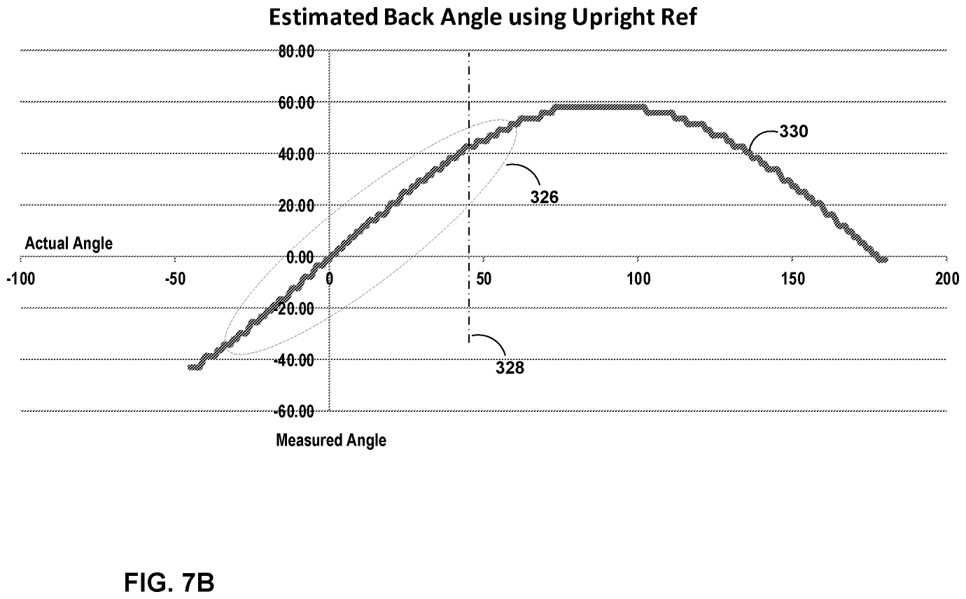

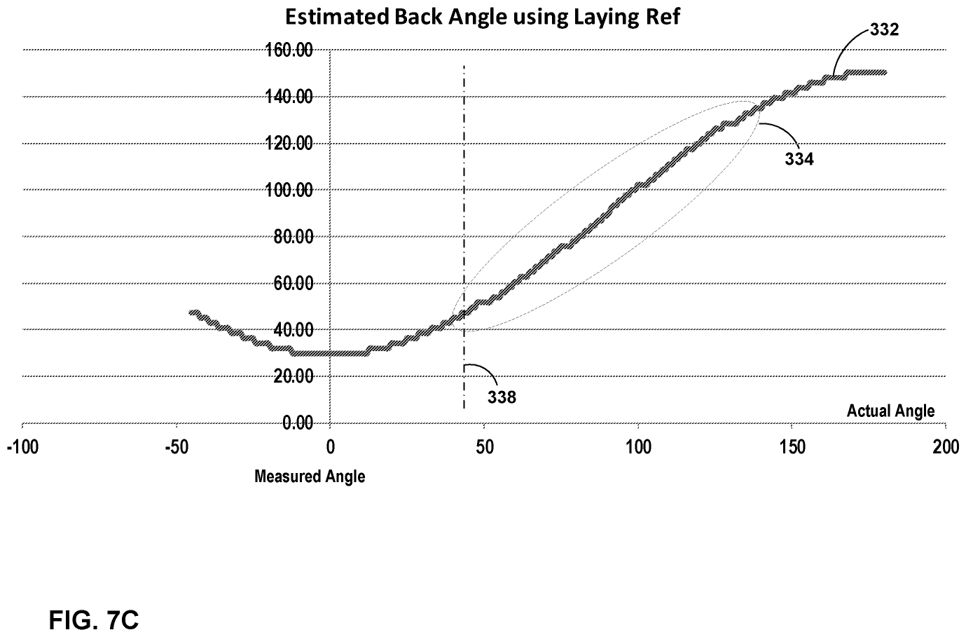

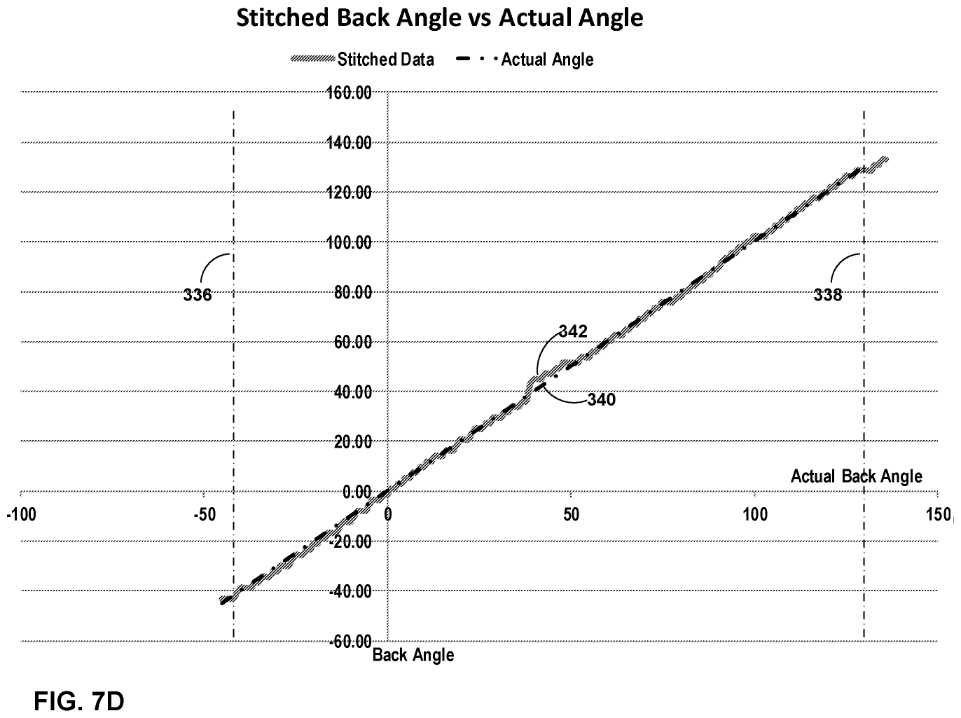

FIG. 7A is a graph illustrating values of an unscaled dot product for the supine (laying) reference template as a function of values of the orientation vector of a medical device. One possible technique for processing circuitry to calculate the angle from the inner-product (cosine) resultant, is to use an arc-cosine function, or a lookup table containing the arc-cosine function. Such an approach may be less accurate for lower resolution accelerometer data, like the 8-bit or 9-bit data in some low power 3-axis accelerometers. At small angles 322, the cosine function 320 is flatter and the resulting angle resolution may be poor. For angles between 45 and 135 degrees, the portion 324 of cosine function 320 is at the most linear and has the greatest slope. Calculating angles between approximately 45 and 135 degrees may be more accurate than for smaller angles, e.g. between −45 and +45 degrees.

FIG. 7B is graph illustrating the relationship between an estimated back-angle determined using an upright reference template and actual measured back-angle values over a range of back-angle values. In FIG. 7B actual measured back-angle values correspond to the sensor orientation vector. For angles between −45 and +45 degrees (328) the dot product 330 of measured orientation angle of the sensor to the upright reference template is in the most linear portion 326.

FIG. 7C is a graph illustrating the relationship between an estimated back-angle determined using a laying reference template and actual measured back-angle values over a range of back-angle values. As with FIG. 7B in FIG. 7C actual measured back-angle values correspond to the sensor orientation vector. For angles between +45 (338) and 135 degrees the dot product 332 of measured orientation angle of the sensor to the upright reference template is in the most linear portion 334.

FIG. 7D is a graph illustrating the near linear estimated back-angle versus actual measured back-angle values determined using an upright reference template for the lower part of the range and a laying reference for the upper part of the range. To cover the entire range of possible patient back-angles, the two position angle approach uses two different orthogonal calculated body position references. When the measured angle values indicate that the angle between the device orientation vector and the reference template is greater than a threshold value, e.g., >45 degrees, the reference is switched to the other orthogonal reference. Switching between the two reference templates, essentially stitches together the linear parts of the curves from FIGS. 7B and 7C to produce a near linear angle estimate from −45° (336) to 135° (338). In FIG. 7D, the stitched together data is shown by curve 342 and the actual angle is shown by curve 340.

As described above in relation to FIG. 6, to simplify the calculations for a less complex processor, such as processing circuitry 80 depicted in FIG. 2, the results of the dot product calculation may be scaled to values that are not in degrees. For example, for an 8-bit device, possible values range from zero to 256. To cover a 360 degree range of angles, the calculation results may be scaled to increments within the 256 range of values. That is, 360/256=1.4 degrees per step. Processing circuitry 80 may perform body angle calculations using these re-scaled values, rather than performing more complex calculations to convert to 360 degrees. As describe above in relation to FIGS. 1-3, simplified calculations may result in reduced power consumption and longer battery life. A medical device, e.g. IMD 10 depicted in FIG. 1, may transmit the scaled values to external device 12. During post-processing, external device 12 may re-scale the numbers back up to their desired degree values.

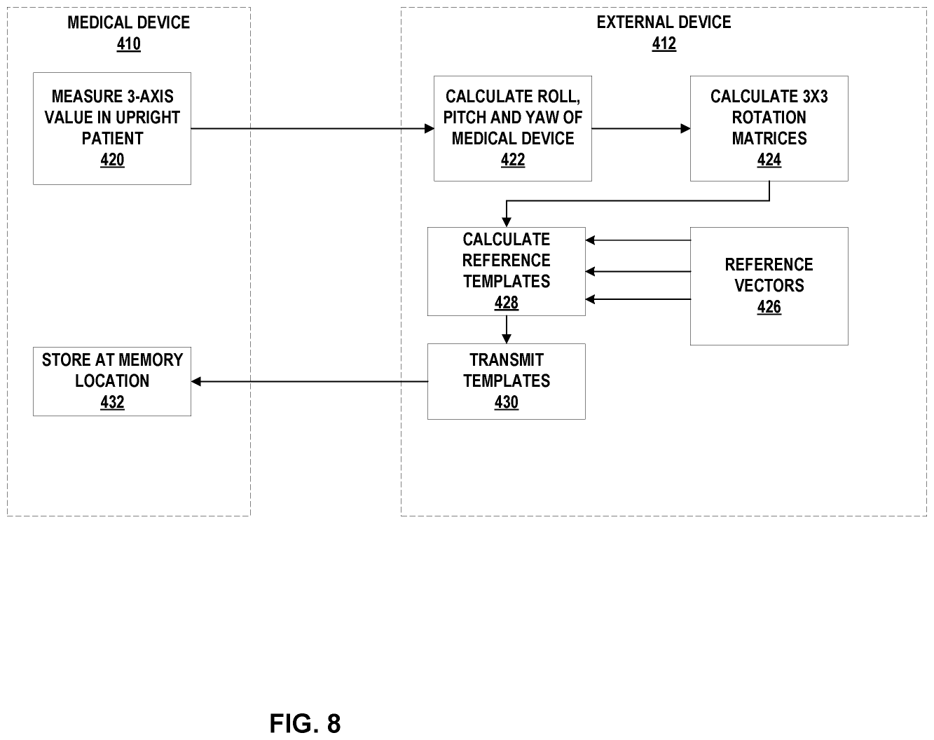

FIG. 8 is a block diagram illustrating an example technique to determine one or more reference templates by an external computing device and store the reference templates in a medical device. Medical device 410 and external device 412 include functions and features similar to IMD 10 and external device 12 described above in relation to FIGS. 1-4.

During compensation, a patient, such as patient 2 depicted in FIG. 1, may be placed in a predetermined compensation reference posture. As described in relation to FIGS. 5A-5C, the upright posture may allow for simplified compensation. For the upright posture processing circuitry of medical device 410 may receive reference templates from external device 412 based on transmitting a single orientation vector to external computing device 412. In other examples, a patient may find it difficult to be placed in an upright posture, especially after an implant surgery. Therefore, other compensation postures may be used.

Processing circuitry of medical device 410 may receive outputs of a sensor that is responsive to a gravitational field, such as a 3-axis accelerometer. The processing circuitry may determine an orientation vector of the sensor relative to a gravitational vector of the gravitational field based on the outputs of the sensor (420). An example gravitational vector is gravity vector 300 depicted in FIG. 6 and an example orientation vector includes orientation vector 205A depicted in FIGS. 5A and 5B. The processing circuitry may transmit to an external computing device, such as external device 412, the orientation vector of the sensor.

Processing circuitry of external device 412 may calculate the yaw, pitch and roll of medical device 410 to determine the orientation vector of the sensor while patient 2 is in the predetermined compensation reference posture (422). Processing circuitry, such as processing circuitry 140 depicted in FIG. 3, may calculate the 3×3 rotation matrices as described above in relation to FIGS. 5A-5C for Ry (yaw), Rx (pitch) and Rz (roll) (424).

Depending on the approach used to determine posture or body angle, i.e., the reference template approach, the two position angle approach or the threshold approach, external device 412 may calculate reference templates (428) using one or more uncalibrated ideal reference vectors (426), such as the upright vector (0,1,0). In one example, medical device 410 may be configured to determine that body position of patient 2 is nearest to one of five orthogonal scaled posture templates calculated by the external device 426 from five ideal unscaled reference vectors: upright (0,1,0), supine (0,0,1), prone (0,0, −1), left side (1,0,0), or right side (−1,0,0). In other examples, medical device 410 may be configured to determine that the body position of patient 2 is nearest to one of ten postures, as listed above in relation to FIGS. 5A-5C. For ten reference templates, external device 412 may include ten ideal reference vectors (426). In some examples, medical device 410 may be configured to convert additional ideal reference vectors to configure medical device 410 to determine additional postures. In other examples, medical device 410 may be configured to determine the patient's body angle based on back-angle and side-angle. For the two position angle approach of back-angle and side-angle, external device 426 may convert three orthogonal ideal reference vectors of upright (0,1,0), supine (0,0,1) and right side (−1,0,0) as described above in relation to FIG. 6 (426) and then provide scaled and converted reference vectors for upright, supine and right side to medical device 410.

Processing circuitry of external device 412 may calculate a plurality of reference templates from ideal reference vectors, e.g. five, ten, or three, or some other number of reference templates, depending on the approach (428). The processing circuitry may transmit the reference templates to medical device 410, e.g. using telemetry circuitry 146 depicted in FIG. 3 (430).

Processing circuitry of medical device 410 may receive the plurality of reference templates from the external computing device 412 and store the plurality of reference templates at a memory location operatively coupled to the processing circuitry (432). An example of a memory location may include memory location 82 coupled to processing circuitry 80 as depicted in FIG. 2.

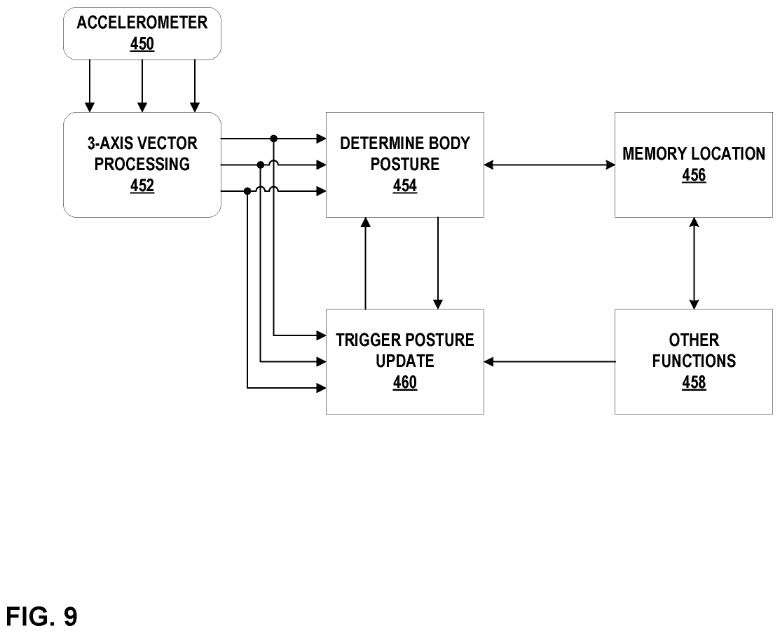

FIG. 9 is a block diagram illustrating example functions performed to determine patient posture according to one or more techniques of this disclosure. The determination of patient posture 454 includes one or more of the reference template approach, the threshold approach and the two position angle approach for determining body angle. The determination of body angle 454 may be done by processing circuitry of IMD 10, a medical device attached to a patient via straps or other means, external device 12 or any of server 164 and computing devices 170, as depicted in FIG. 4. The functions of FIG. 9 are depicted as separate blocks, but may all be performed by a single processor, such as processing circuitry 80 in IMD 10, or distributed to other processing circuitry or hardware of one or more other devices described herein. In other words, a hardware circuit may be used to directly calculate the patient body angles and positions to offload the calculations from the processor. In some examples firmware may load a register with a reference accelerometer vector, or other values, and have the hardware calculate a dot-product between the accelerometer orientation vector and a reference template. Exclusion of a normalizing division may further simplify the calculations for this dot-product implementation. The division may instead be approximated as a power of 2 shift by the firmware. A normalizing division step may be included in the post-processing of data.

A processor, such as processing circuitry 80 of IMD 10 depicted in FIG. 2, may receive sensor outputs from accelerometer 450 and perform 3-axis vector processing 452, as described above in relation to FIGS. 5A-6. Three-axis vector processing 452 may also include amplifying, filtering, and interpreting voltage or current output signals as an orientation vector for accelerometer 450. Functions of accelerometer 450 and 3-axis vector processing 452 may correspond to functions of orientation and activity state circuitry 86 and orientation sensor 87 as described above in relation to FIG. 2.

In some examples, the three native axis signals from the accelerometer may be combined at four different digital summers. Three of the digitally summed channels may be aligned with x, y, and z axes, and may be low-pass filtered at 0.5 Hz and decimated, for slowly changing orientation measurements. One of the channels may be high-passed filtered at 1 Hz and used for the activity measurements. In other examples the vector processing circuitry 452 may be configured to create three activity channels, one for each axis. The vector processing 452 may generate three orientation channels by choosing to use analog summers to create the 3 orientation axes.

The patient's posture, or body angle, determined by processing circuitry 80 (454) may be stored at memory location 456, which may be included in memory 82 of FIG. 2, along with timestamp information, activity level information physiological information gathered by other sensors, as well as other information. Memory location 456 may also include instructions for how to perform posture determination functions, as well as other functions 458 performed by, for example, IMD 10.

In some examples, a medical device may determine posture either continuously or at regular timed intervals. However, posture calculations consume power, such as from power source 90 depicted in FIG. 2. More posture calculations may reduce battery life. However, large timed intervals between posture calculations may miss intermediate postures that may be helpful, for example, to a clinician. To minimize power consumption, yet collect any intermediate postures, a trigger posture update 460 function may be implemented, e.g., by processing circuitry 80. In some examples, a medical device may not perform a posture calculation unless the patient moves. For example, during sleep, working at a desk, or other activities, a patient may remain in a single posture for an extended time.

In other examples, a patient's activity level may be high, such as during exercise, walking, and other activities. If the activity level is high, repeated posture calculations may not be as accurate. Trigger posture update 460 may only signal a posture calculation when the activity level is below a threshold, for example.

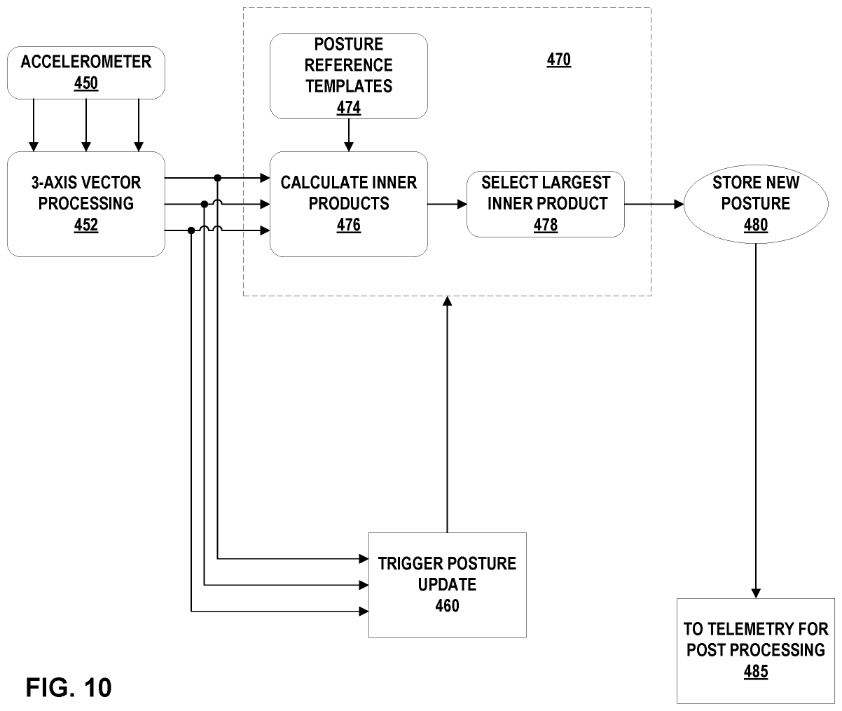

FIG. 10 is a block diagram illustrating an example technique for determining patient posture using a plurality of reference templates. Accelerometer 450, 3-axis vector processing 452 and trigger posture update 460 are the same as described above in relation to FIG. 9.

Determine patient posture 470 corresponds to determine patient posture 454 as depicted in FIG. 9. In the example of FIG. 10, to determine patient posture 470, a processor may retrieve a plurality of posture reference templates 474 from a memory location, calculate inner products 476 and select largest magnitude inner product 478. In other examples that may use a different angular distance metric other than the dot, or inner, product, the processing circuitry may select, for example, the angular distance metric with the smallest magnitude, i.e. the minimum magnitude.

As described above, in relation to FIGS. 5A-5C and FIG. 9, the determination of a patient's posture may use a plurality of reference templates 474. Some examples may include five, ten or more reference templates. More reference templates provide a less coarse, more granular determination of posture, but require additional calculations, and therefore may consume more power. In some examples, the set of reference templates may define a sitting/sleeping angle by creating laying references at five, 10 or some other degree of body angle increments such as from −30 to 60 degrees. In other words, the orientation of the patient's body part is an orientation of the patient's chest and a number of reference templates may define a set of sleeping angles. as one example. For example, the set of reference templates may include a set of reference templates indicating the orientation of the patient's chest including: −30, −20, −10, 0, 10, 20, 30, 40, 50 and 60 degrees.

For each triggered posture update signal 460, the processor may calculate an angular distance metric, such as inner product 476, based on the received signals from accelerometer 450 via 3-axis vector processing 452. The received signals indicate the posture vector of accelerometer 450 relative to the gravitational field. For the calculate inner products function 476, a processor may perform an inner product (i.e. dot product) calculation of the sensor orientation vector with each of the plurality of reference templates 474

A processor may select largest inner product 478 function based on the output of the scalar results of the plurality of inner product calculations. As described above, the cosine function is at a maximum when the angle between two vectors is zero. Therefore, the scalar value of the dot product between two vectors is at a maximum when the angle between the two vectors is zero. A reference template, such as the upright reference template, may be a vector that describes the orientation vector a device when the patient is upright. When the device orientation vector is pointing in substantially the same direction as reference template direction, the angle between the orientation vector and the reference template direction is small, i.e. the cosine between the orientation vector and the reference template direction is large. A dot product for the upright reference template that is larger than the dot products for other reference templates (e.g. right side up, face, etc.), may indicate that the patient is closest in angle to upright. In other words, the processor determines which scalar dot product is largest and the one of the plurality of postures associated with the largest of the plurality of dot products as the current posture of the patient.

The processor, e.g., processing circuitry 80, may store the updated posture at a storage location (480). The processor may also store other information, such as a timestamp, associated with the posture.

As described above in relation to FIGS. 5A-8, to further simplify calculations, the steps of FIG. 10 may be accomplished by using raw, scaled values rather than converting the values to angles in degrees. A processor may transmit information stored during step 480 for post-processing (485), such as to an external device, which may convert the raw values for use in graphs, tables and other uses.

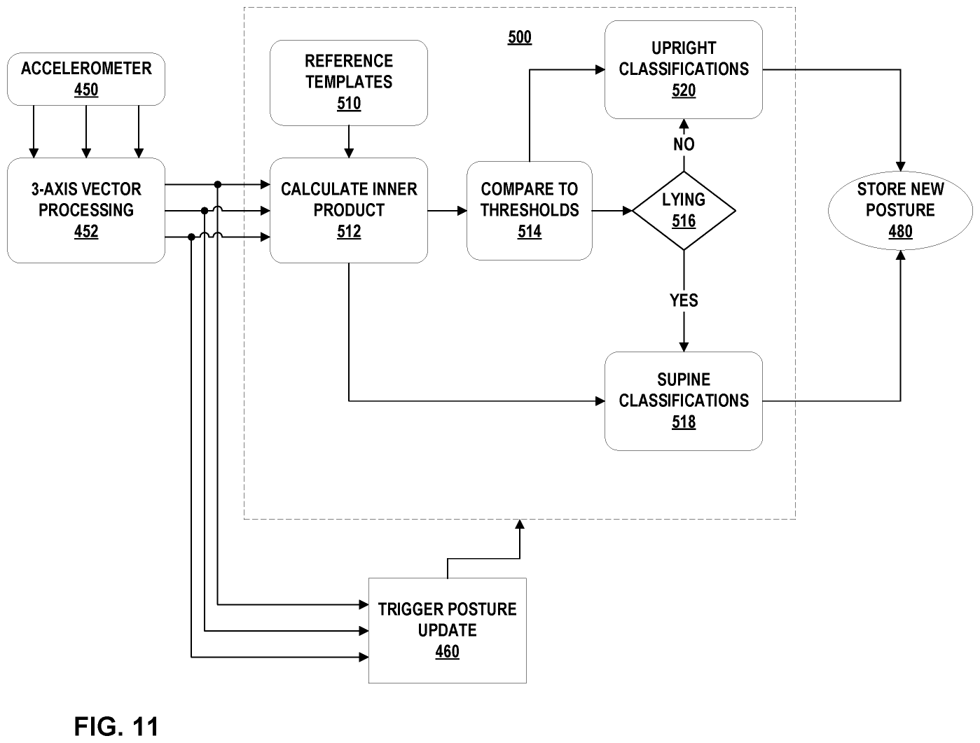

FIG. 11 is a block diagram illustrating an example technique for determining patient posture using a threshold classification approach. The threshold classification approach may also be referred to as a heuristic approach. The example of FIG. 11 may be executed by a processor in a medical device, such as processing circuitry 80 IMD 10, or an external computing device, such as processing circuitry 140 of external device 12, depicted in FIG. 4. The threshold approach may also be executed during post-processing 112, as described above in relation to FIG. 3. Accelerometer 450, 3-axis vector processing 452, trigger posture update 460, and store new posture 480 are the same as described above in relation to FIGS. 9 and 10.

Determine patient posture 500 corresponds to determine patient posture 454 as depicted in FIG. 9. In the example of FIG. 11, to determine patient posture 500, a processor may retrieve a plurality of posture reference templates 510 from a memory location. In some examples, for the threshold approach, only the upright reference template (0,1,0) may be used.

For each triggered posture update signal 460, the processor may calculate inner product 512 based on the received signals from accelerometer 450 via 3-axis vector processing 452. As described above in relation to FIG. 9, the inner product yields the product of the cosine of the angle between the sensor posture vector and the magnitude of each vector.

The processor may compare the scalar dot product to a set of thresholds (514). In some examples the threshold comparison may indicate that the patient is in a lying down posture (516). If the patient is in a lying down posture (YES of 516) the processor may use the heuristic sign (positive or negative) of the posture vector signals (ax, ay, az) and magnitude comparisons to classify which lying down posture that patient is in. For example, supine, prone, right-side up or left-side up (518).

If the patient is not in a lying down posture (NO of 516), the processor may compare the scalar dot product to one or more thresholds to determine which of the upright postures the patient is in (520). Some example upright postures may include standing upright, upside down or sitting. For either lying down or upright postures, the processor may store the information at a memory location (480).

The determine posture (500) steps may also be described in terms of a logic flow, similar to a computer program. The values in the below logic flow description (e.g. 0.5, 0.85) are for illustration purposes only. Other thresholds may be equally valid depending on the compensation, programming, selected hardware, accelerometer characteristics and other parameters. An example of heuristic logic flow steps may include:

If {(x, y, z)●Upright(X, Y, Z)≥0.85}⇒Upright

If {0.5≤(x, y, z)●Upright(X, Y, Z)<0.85}⇒Sitting

If {(x, y, z)●Upright(X, Y, Z)≥−0.707}⇒Upside Down

If {Not Upright, Sitting, or Upside Down}⇒Laying
If Laying⇒Split into Left Side Up, Right Side Up, Face Down and Face Up
If {Laying and z<−0.6}⇒Face Down
If {Laying and z>0.6}⇒Face Up

If Not Face Down or Face Up⇒Check if LSU or RSU
If {Upright X>0 and Upright Y≤0}⇒0 to 90 Quadrant (Qu1)⇒
   If {|x|>|y| and x>0}⇒RSU
   If {|x|>|y| and x≤0}⇒LSU
   If {|y|>|x| and y>0}⇒RSU
   If {|y|>|x| and y≤0}⇒LSU
If {Upright X>0 and Upright Y>0}⇒90 to 180 Quadrant (Qu2)⇒
   If {|x|>|y| and x>0}⇒LSU
   If {|x|>|y| and x≤0}⇒RSU
   If {|y|>|x| and y>0}⇒RSU
   If {|y|>|x| and y≤0}⇒LSU

Where:
LSU=Left-side Up,
RSU=Right-side Up,
   |x|=magnitude of the X-axis value of the posture vector
(x, y, z)●Upright(X, Y, Z)=dot product of the posture vector and the upright reference vector.

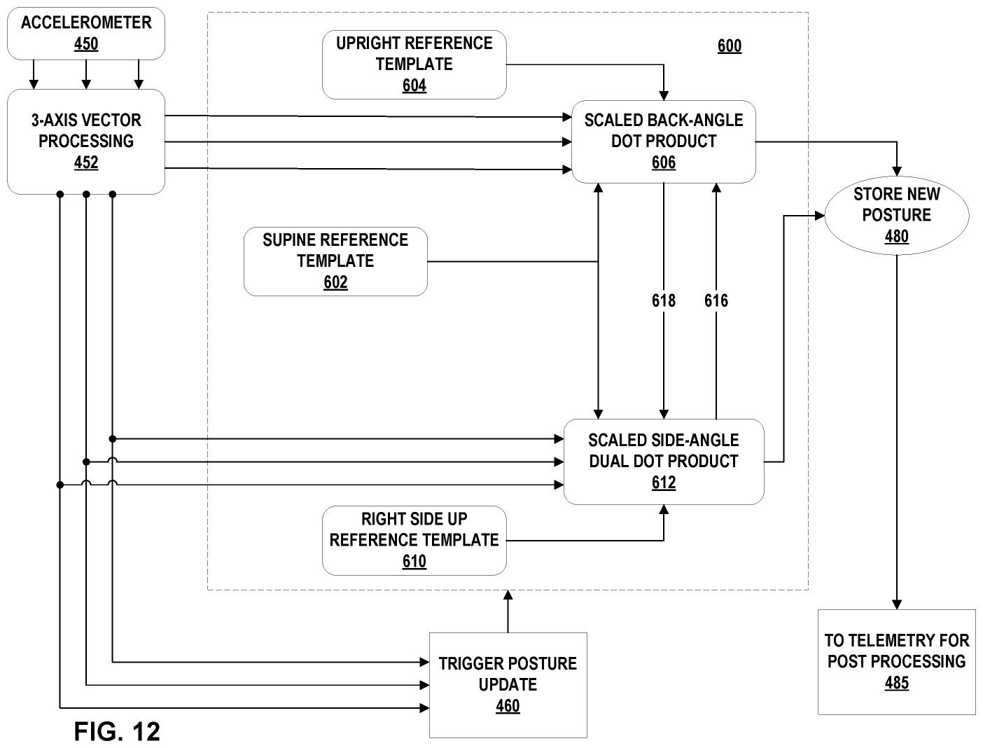

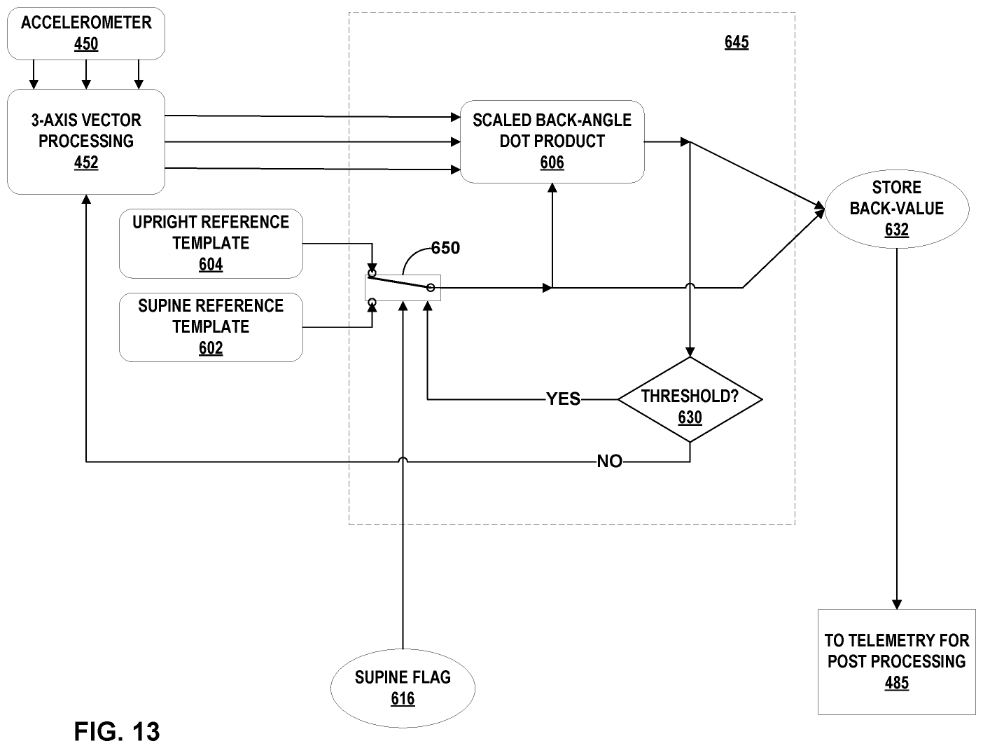

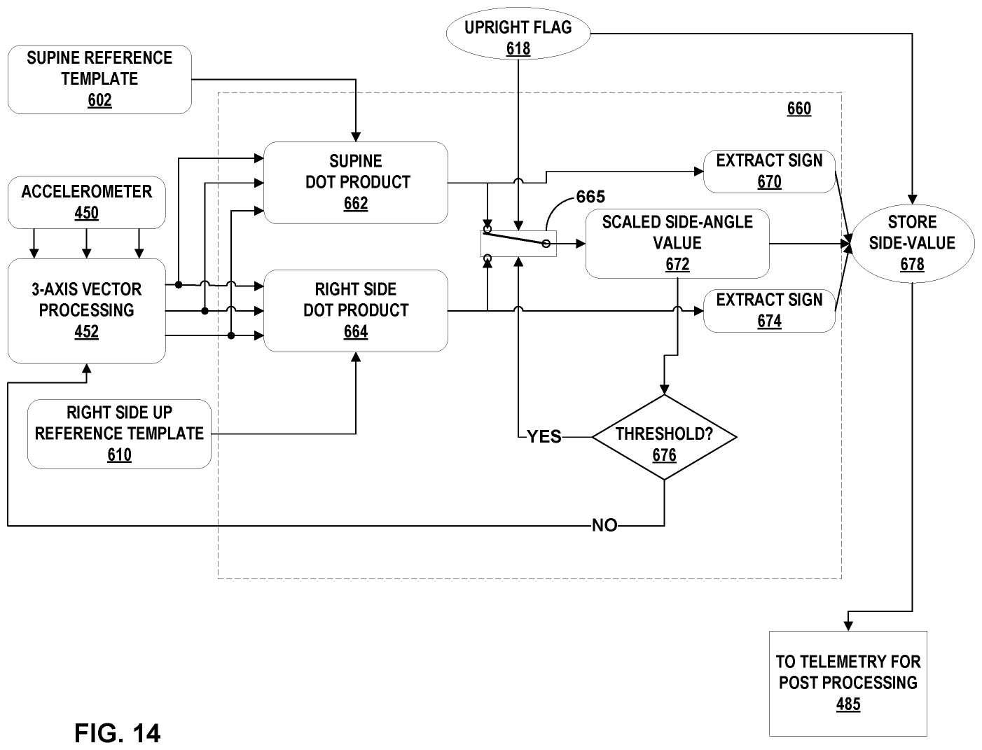

FIG. 12 is a block diagram illustrating an example technique for determining patient posture by determining a scaled back-angle and scaled side-angle. FIG. 12 provides an overview of the two posture angle approach, while FIG. 13 provides details of the back-angle determination and FIG. 14 provides details of the side-angle determination. Accelerometer 450, 3-axis vector processing 452, trigger posture update 460, store new posture 480 and post-processing 485 are the same as described above in relation to FIGS. 9 and 10.

Determine patient posture 600 corresponds to determine patient posture 454 as depicted in FIG. 9. In the example of FIG. 12, to determine patient posture 600, a processor, e.g., processing circuitry 80 of IMD 10, may determine a value representative of a body angle of a patient. As described above in relation to FIG. 6, the processor may store a first reference template, e.g. the upright reference template (604) and a second reference template, e.g. the supine reference template (602) at a memory location. The upright reference template is orthogonal to the supine reference template.

The processor may receive outputs of accelerometer 450 via 3-axis vector processing 452. As described above, the processor, or other circuitry, may determine an orientation vector of accelerometer 450 relative to a gravitational vector of the gravitational field, based on the outputs of accelerometer 450.

The processor may select one of the upright reference template or the supine reference template for determining the posture value based on the orientation vector (606). In other words, the processor may determine the scaled back-angle dot product by selecting either the upright or the supine reference template, depending on which reference template is more different from the sensor orientation vector. As described in relation to FIG. 7D, to further simplify the calculations, the scaled back-angle may be limited to 8-bit or 9-bit calculation, or a similar scaling limit. The scaled value may be considered the value representative of the body angle of the patient based on a dot product of the orientation vector and the selected reference template.

In some examples, as described above in relation to FIG. 6, when the orientation vector is less than approximately forty-five degrees from a reference template, the processor may switch to the orthogonal reference vector for improved accuracy. In other words, the processor may determine whether a difference between a direction of the orientation vector and a direction of a reference template satisfies a threshold, based on the dot product of one reference template with the orientation vector. If less than the threshold, e.g. approximately forty-five degrees, the processor may select the other reference template.

In some examples, back-angle dot product (606) may also depend on which reference template was used to determine the side-angle dot product (612). The back-angle dot product (606) calculation may receive information about which template was used by the side-angle dot product (612) via supine flag 616. The side-angle dot product (612) passes the supine flag 616 to the back-angle dot product (606) when the side-angle dot product switches to the supine reference template (602). The supine flag 616 is to account for a blind-spot in the back-angle measurement. The blind-spot occurs because the back-angle dot product (606) cannot detect a posture transitions where the patient lies down directly from upright to either the right-side up posture or the left-side up posture. The supine flag signals the back-angle block to switch to the upright reference template (604).

Another way to describe the back-angle calculation is that only one inner-product is required in any patient body angle measurement cycle, because the back-angle only has to represent the angle of the long-axis of the chest with respect to the horizontal plane. The back-angle switches between two references templates, the measured upright reference template and the calculated supine reference template. The implementation of FIG. 12 may determine back-angles from −45 degrees to 135 degrees.

The scaled back-angle dot product (606) passes the upright flag 618 to the scaled side-angle dot product (612). The upright flag 618 disables the face-down adjustment of the side-angle measurement and switch to only using the right-side up reference template (610), so that the side-angle value represents only a side-to-side-angle measurement when upright.

The processor may also store a third reference template, i.e. right-side up reference template (610). Right-side up reference template (610) is orthogonal to both the upright reference template and the supine reference template, as described above in relation to FIG. 6. The processor may calculate the side-angle dot product (612) by calculating a dot product of the orientation vector with both the supine reference template (602) and right-side up reference template (612). Side-angle dot product (612) may be considered a dual dot product, in contrast to back-angle dot product 606. In other words, for the side-angle, the processor may perform two inner-product calculations to distinguish face-up from face-down simultaneous to distinguishing left side versus right side. The implementation of FIG. 12 may measure side-angles from 0 to −180 degrees, representing all face down angles, and 0 to 180 degrees, representing all face-up angles. In other examples, the third reference template may be a left-side up template.

As with the back-angle, the side-angle dot product may be a scaled value. The side-angle dot product may be considered a value indicative of the side-angle, rather than an actual angle in degrees. In some examples, the model or type of accelerometer 450 and how the accelerometer is configured, or trimmed, e.g. during manufacturing, may also impact the calculations of back-angle and side-angle. The configuration and other parameters of accelerometer 450 may need to be factored into the back-angle and side-angle calculations, as well as factored into the conversion and scaling calculations during post-processing.

In some examples, the side-angle dot product (612) may also depend on which reference template the processor used to determine the back-angle dot product (606). Side-angle dot product (612) may receive information on which reference template the processor used for the back-angle via upright flag 618.

As described above in relation to FIG. 10, the processor may store the body angle information at a memory location (480). The processor may be further configured to determine an assigned posture value of the patient based on the back-angle and the side-angle. In some examples IMD 10, depicted in FIGS. 1 and 4, may determine the assigned posture value and may take action based on the posture value. For example, IMD 10 may signal another sensor or therapy device to take a measurement, e.g. a pressure reading, or to deliver therapy. In other examples, IMD 10 may transmit body angle information to an external computing device for post-processing (485).

FIG. 13 is a block diagram illustrating additional details of an example technique for determining a scaled back-angle, such as by a medical device. Back-angle calculation 645 includes additional details from the back-angle determination of FIG. 12. Accelerometer 450, 3-axis vector processing 452, upright reference template 604, supine reference template 602 and supine flag 616 are the same as described above in relation to FIGS. 9, 10 and 12.

Back-angle calculation 645 includes an output from template switching function 650 to the scaled back-angle dot product 606. Switching function 650 may be implemented, for example by logic circuitry, software or firmware instructions stored at a memory location, or by circuit components such as on an integrated circuit. A processor may switch the output from switching function 650 between upright reference template 604 and supine reference template 602. As described above in relation to FIG. 12, the processor may calculate the scaled back-angle dot product (606) between the sensor orientation vector and the selected reference template. One way to describe the scaled back-angle dot product, as used to simplify calculations, is to consider the dot product as divided by a scale factor. In one example, the scale factor (S.F.) may be the value 64 (64=2⁶) to bring the dot product down to an 8-bit number for ease of storage purposes. In some examples signals may be further coerced into a range to avoid having out of range numbers during activity. In one example, the signals may be coerced to be within a range of ±28. In other examples, the range may be different depending on the choice of accelerometer, type of processor and other factors. Once the processor calculates the scaled back-angle dot, the processor may pass the values to the final angle formula calculations and stored (632). For post-processing (485) the same S.F. may be used convert the scaled values.

Before continuing to get the next data value, the scaled back-angle dot product (606) output may be tested against a threshold (630) to determine if the angle between the orientation vector from 3-axis vector processing (452) and the selected reference has now exceeded the threshold (e.g. a value indicative of 45°). If true (YES of 630), then the processor may switch reference template for the back-angle dot product (606) to the other reference template, e.g. control switching function 650 to switch to the other reference template. The processor may retrieve next set of accelerometer 450 output values, for example, when triggered by the trigger posture update function (460) described in relation to FIG. 9. If not true (NO of 630), then the processor may continue by retrieving the next set of accel values using the same reference template.

For lying down postures (i.e. positions where the back-angle <45 degrees, as depicted in FIG. 6), the back-angle dot product (606) is measured using upright reference template (604). For lying down postures, the negative values of back-angle indicate up-side down orientations, and positive values indicate heads-up orientations. In other examples, the assignments may be different, e.g., for lying down postures the positive values of back-angle may indicate up-side down.

For upright postures (orientations of the chest >45 degrees), the back-angle dot product (606) is measured using supine reference template (602). For upright postures, negative values indicate leaning forward and positive values indicate leaning back. The implementation of FIGS. 12 and 13 may therefore measure back-angles from −45 degrees to 135 degrees. Therefore, the switching between reference channels is done up front, prior to calculating the inner-product and the back-angle dot product need only calculate one dot product.

In some examples, the actual implementation in a medical device may constrain the range to a value between 135 degrees. For example, the range may be between −25 degrees to 135 degrees. In a limited range example, the processor may flag all the values less than −25 degrees or just replace the values less than −25 degrees as being equal to −25 degrees. Range limitation may depend on a variety of factors including type of accelerometer, circuit configuration, power consumption limits, and so on.

The next paragraphs describe additional detail on the back-angle calculation 645, including related equations. As discussed in general terms above, when calculating the back-angle, a partially scaled inner-product may be calculated using one of the calculated reference values stored in memory. Instead of dividing by the exact amplitude of the signal, a power-of-2 division may be used to simplify calculations. For example, a scale factor of 64 results in a resolution of 1.45 degree/step. The scaled inner product for both back-angle and side-angle is according to the following equation:

scaled inner product=(ax_measured*ax_reference+ay_measured*ay_reference+az_measured*az_reference)/2ⁿ (30)

where (ax_measured, ay_measured, az_measured) is the sensor orientation vector. The value of n may be selected as a scaling division to keep the output signal within range (i.e. coerce the signal). Also, the processor may switch references when the scaled dot product satisfies a threshold. In some examples, the threshold is a scaled value indicative of 45 degrees.

As described below, the back-angle is the converted back-angle in degrees, such as after post-processing. Values that may be scaled and stored in a medical device with a less complex processor, such as IMD 10 described above in relation to FIG. 2. include the scale factor (S.F.) and Scaled_B_Value, which is the output of scaled back-angle dot product (606). The side-angle calculations depend on what part of the range the back-angle is in. For example:

Back-angles from 45 to 135 degrees:
When using the Laying Reference in the Back-angle calculation:

Back_Angle=[90−(S.F.)*(Scaled_B_Value)], (31)

Where:

S.F.=[(Number Bits per G)/38]*(2\hat{ }n/32) (32)

and n is the number of bits the processor shifts left to scale the inner-product result. For example, if there are 42 bits/G and n=6⇒

S.F.=(42/38)*(64/32)=2.2) (33)

Also for n=5, ⇒S.F.=1.1 (34)

For Back-angles from −45 to 45 degrees:

When using the Upright Reference in the Back-angle calculation, the

Back_Angle=[90−(S.F.)*(90*(1/(S.F.)−(Scaled_B_Value)] (35)

FIG. 14 is a block diagram illustrating additional details of an example technique for determining a scaled side-angle by a medical device. Side-angle calculation 660 includes additional details from the back-angle determination of FIG. 12. Accelerometer 450, 3-axis vector processing 452, right-side up reference template 610, supine reference template 602 and upright flag 618 are the same as described above in relation to FIGS. 9, 10 and 12.

For side-angle calculation 660 block the processor, e.g., processing circuitry 80 of IMD 10, executes two inner products between the sensor orientation vector and reference templates, i.e. the supine dot product (662) and the right-side dot product (664). As depicted in the example of FIG. 14, the supine dot product (662) uses supine reference template (602) and the orientation vector, while the and the right-side dot product (664) uses the right-side up reference template (610) and the orientation vector. As described above in relation to FIGS. 9 and 12, the sensor orientation vector is an output of 3-axis vector processing 452.

After the processor determines the two inner products switching function 665 selects the desired inner-product, whether calculated using the supine reference template (602) or the right-side up reference template (610). To avoid having out of range numbers during activity, in some examples the processor may coerce the output signals of signals supine dot product (662) and right-side dot product (664) to a specific range. Once the processor selects the dot product, the dot product values are passed on to the final side-angle formula calculations (672) and stored (678). For post-processing (485) scale factors (e.g. S.F.) may be used convert the scaled values.

The sign for supine dot product (662) may be extracted and stored (670) along with the scaled side-angle value 672. Similarly, the sign of the right-side dot product (664) may be extracted and stored (674) along with the scaled side-angle value 672. During post-processing, the sign of the dot product may be used to convert the scaled values of the side-angle dot product 672 to degrees, for example. The sign of supine dot product (662) is used to determine face up (positive) or face down (negative). Similarly, the sign of the right-side dot product (664) is used to determine right-side up (positive) or left-side up (negative).

Similar to the back-angle calculation described above, before continuing to get the next data value, the processor may test selected scaled dot product against a threshold (676) to determine if the angle between the signal and the reference template has exceeded a threshold, such as approximately 45°. If true (YES of 676), then the processor may switch the reference template for side-angle calculation 660 to the other reference template. The processor may retrieve the next set of orientation vector values from 3-axis vector processing 452 and use the other reference vector. If not true (NO of 676), then the processor may continue by retrieving the next set of orientation vector values and determine the next dot product using the same reference template.

Additionally, for the side-angle calculation 660 it is desirable to distinguish face up from face down and left-side up from right-side up independently, for any side-angle over the entire ±180-degree range. Because face-up vs face down can only be distinguished by using the sign of the inner-product with the supine reference (602) and left-side-up and right-side-up can only be distinguished by using the sign of the inner-product with the right-side-up reference (610), the processor calculates both inner-products in parallel for any given side-angle measurement. Therefore, the switching between the two reference channels at switching function 665 is done after the calculation of the two inner-products.

Side-angle calculation 660 uses the right-side-up reference (610) for side-angles between −45 Degrees and 45 Degrees, between 135 degrees and 180 degrees, and between −135 degrees and −180 degrees. Similarly, side-angle calculation 660 uses the supine reference (602) for side-angles between 45 degrees and 135 degrees, and −45 degrees and −135 degrees. The implementation of FIGS. 12 and 14 may therefore measure side-angles from 0 to −180 degrees, representing all face down angles, and 0 to 180 degrees, representing all face-up angles.

The two position angle approach may include some special posture situation checks. During the calculations of these scaled angle values, there may be several special posture situations in which it may be useful to switch the reference template selections to specific states. As described above in relation to FIG. 13, one situation is a blind-spot to transition from an upright posture directly to a left-side up or a right-side up posture. The other is because when the patient is upright, using a supine reference template for side-angles means the side-angle calculation will actually incorrectly be determining the back-angle. The processor may use upright flag 618 and supine flag 616 for these situations.

The back-angle threshold (630) test depicted in FIG. 13 is blind to a change from an upright posture directly to left or right-side posture. An upright-to-side transition may allow the inner product calculation to remain below threshold, because a twist to the side keeps the angular distance between the sensor position vector on the side and the supine reference template (602) high. To work around this blind spot, the fact that the side-angle also makes a reference template transition for the same upright-to-side transition is used to force the reference for the back-angle measurement to the upright reference template (604) by using the supine flag (616).

In the example of side-angle calculation 660 while in an upright posture, the processor uses upright flag 618. As described above when upright, side-angle calculation 660 should not use supine reference (602) to calculate the side-angle, because the processor will be measuring the back-angle instead. To ensure side-angle calculation 660 uses right-side up reference template (610), the processor may check if back-angle calculation 645 used supine reference (602). If so, upright flag 618 forces switching function 665 to the right-side up reference template (610).

The next paragraphs describe additional detail on the side-angle calculation 660, including related equations. As discussed in general terms above, when calculating the side-angle, the processor may include steps to recognize face down from face up by using the sign of the inner-product between the orientation vector and the supine reference template (602). Also, the processor may recognize left side up from right side up using the sign of the inner-product between the orientation vector and right-side up reference template (610). As described below, “Side-angle” is the converted side-angle in degrees, such as after post-processing. Values that may be scaled and stored in a medical device with a less complex processor, such as IMD 10 described above in relation to FIG. 2 include the scale factor (S.F.) and Scaled_S_Value, which is the output of scaled side value (672).

The side-angle calculations depend on what part of the range the side-angle is in. For example, when the processor uses right-side up reference template (610) this indicates the patient is on their back OR stomach AND:

For side-angles between 90 to 135 degrees:
If Face Down is true AND back-angle calculation 645 used upright reference template (604) AND Right Side Up is true:⇒

Side-angle=(S.F.)*[180/(S.F.)−(Scaled_S_Value)] (36)

For side-angles from −90 to −135 degrees:
If Face Down is true AND back-angle calculation 645 used upright reference template (604) AND Right Side Up is false: ⇒

Side-angle=(S.F.)*[−180/(S.F.)−(Scaled_S_Value)] (37)

For side-angles from −45 to 45 degrees:
Otherwise (if Face Down is false OR if back-angle calculation 645 used supine reference template (602) (indicating standing)⇒

Side-angle=S.F.*(Scaled_S_Value) (38)

When the processor uses supine reference template (602), which indicates the patient is on one of their sides AND:

Side-angles from 45 to 90 degrees:
If the Right Side Up inner-product is positive in sign⇒

Side-angle=(S.F.)*(+1)*[90/(S.F.)−(Scaled_S_Value)] (39)

Side-angles from 45 to 90 degrees:
If the Laying reference is being used AND If the Right Side Up inner-product is negative in sign⇒

Side-angle=(S.F.)*(−1)*[90/(S.F.)−(Scaled_S_Value)] (40)

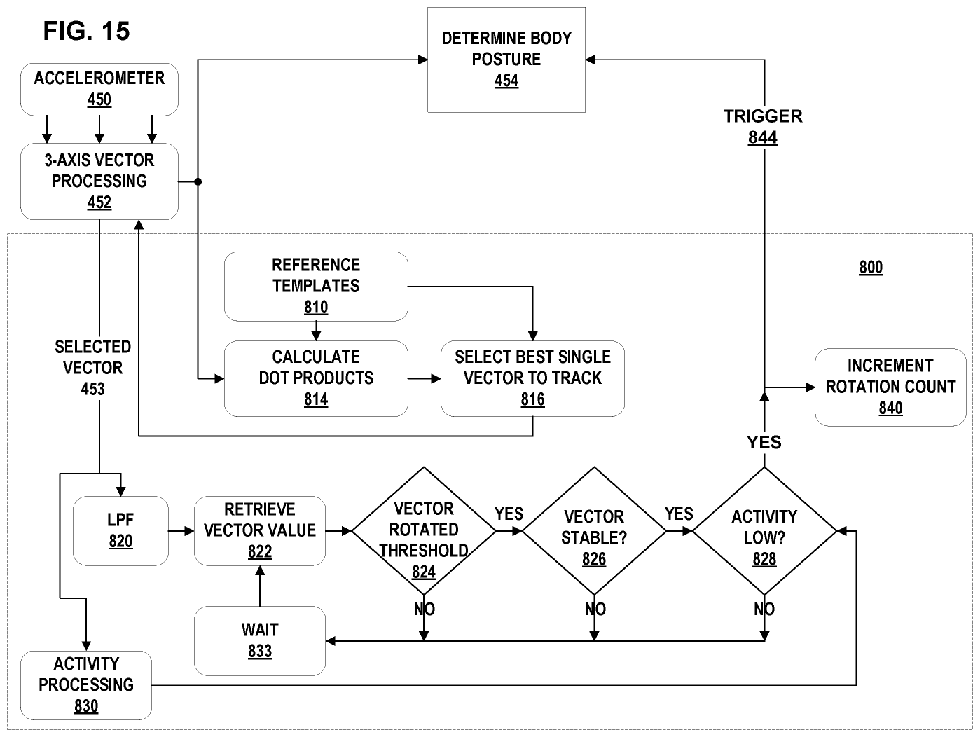

FIG. 15 is a block diagram illustrating an example of a power efficient technique to trigger a medical device to update a patient's posture. The example of FIG. 15 is one technique for triggering a posture update, similar to that described by 460 in relation to FIG. 9. In some examples processing circuitry may execute the steps of FIG. 15. In other examples, some calculations in FIG. 15 may be offloaded to a hardware based processing circuit. Accelerometer 450, 3-axis vector processing 452 and determine posture 454 is the same as described above in relation to FIG. 9.

Posture change detector 800 is a power efficient angle tracking implementation suitable for low power applications, such as within IMD 10 depicted in FIG. 1. Orientation change detector 800 may trigger an update to the orientation and activity state information, for example if a patient changes sleeping positions. Orientation change detector 800 may also be referred to as a posture change algorithm.

Reference templates 810 include combinations of the x, y and z axes, similar to axes depicted in FIGS. 5A-5C. Some examples of arithmetic combinations may include x+y, x+z, x−y, x−z, z−x, y−z, and similar combinations. These combinations of reference vectors may include arithmetic combination of one, two or three axes of the sensor and may be easily calculated, for example, by a hardware circuit implementation.

Posture change detector 800 may select a single reference vector (816) of the plurality of reference vector templates 810. Selecting a single reference vector may also be referred to as selecting a single channel. The single reference vector is selected to be the closest to the current orientation vector of accelerometer 450. By selecting the single reference vector (816) closest to the sensor orientation vector, allows a high sensitivity to posture changes.

Posture change detector 800 may determine the closest reference vector by calculating a dot product (814), as described above in relation to FIGS. 1-14. Calculate dot products 814 function may receive the orientation vector from 3-axis vector processing 452 and determine the closest reference vector, for example by determining the largest dot product. The largest dot product is then selected (816) as the best vector to track 453 for change detection from 3-axis vector processing 452.

In the example in which posture change detector 800 detects a posture change, then posture change detector 800 may increment the rotation counter 840 that indicates patient movement and trigger an updated posture determination (844). In some examples a processor, such as processing circuitry 80 depicted in FIG. 2, may wake up at a predetermined interval (833), such as every second or every two seconds, to check if posture change detector 800 has detected a posture change. In the example in which there is no posture change, then the processor may perform no further posture calculations.

Low pass filter (LPF) 820 receives a selected single-axis vector from a 3-axis vector processing circuit 452 and outputs a filtered signal to threshold comparator 824. In some examples, low pass filter 820 may be similar to the 0.5 Hz low-pass filter as described above in relation to FIG. 9. The low-pass filtered and selected tracking vector 822 is compared against a threshold to recognize when an angle change has indicated a major posture change has occurred.

When the retrieved vector rotation (822) satisfies a threshold (YES of 824) of angle comparator 824, then orientation change detector 800 may verify the stability (826) of the selected low-pass filtered vector. In the example in which the selected low-pass filtered vector satisfies a stability threshold (YES of 826), then orientation change detector 800 may check the patient's activity level (828).

Activity processing 830 receives accelerometer information from 3-axis vector processing 452 and outputs an indication of patient activity to activity threshold detect 828. In some examples, activity processing 830 may include filtering and other signal processing, similar to that described above in relation to FIG. 9. When the patient's activity level is low, for example, when the patient is sleeping (YES of 828), orientation change detector 800 may determine that there has been a posture change.

In the example in which the selected signal vector does not satisfy the posture change threshold (NO of 824), the selected signal vector is not stable (NO of 826) then orientation change detector 800 may wait for the predetermined interval (833), such as when a processor wakes up. Similarly, if the patient's activity level is high (NO of 828), the patient may be walking or otherwise active and orientation change detector 800 may not trigger an updated posture determination.

In one or more examples, the functions described above may be implemented in hardware, software, firmware, or any combination thereof. For example, the various components of FIG. 4, such as IMD 10 and external device 12 and server 164 may be implemented in hardware, software, firmware, or any combination thereof. If implemented in software, the functions may be stored on or transmitted over, as one or more instructions or code, a computer-readable medium and executed by a hardware-based processing unit. Computer-readable media may include computer-readable storage media, which corresponds to a tangible medium such as data storage media, or communication media including any medium that facilitates transfer of a computer program from one place to another, e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media, such as memory 142 depicted in FIG. 3, can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transient media, but are instead directed to non-transient, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Instructions may be executed by one or more processors, such as one or more DSPs, general purpose microprocessors, ASICs, FPGAs, or other equivalent integrated or discrete logic circuitry. Accordingly, the term “processor,” as used herein, such as processing circuitry 140, may refer to any of the foregoing structure or any other structure suitable for implementation of the techniques described herein. In addition, in some aspects, the functionality described herein may be provided within dedicated hardware and/or software modules configured for encoding and decoding, or incorporated in a combined codec. Also, the techniques, such as orientation change detector 800 could be fully implemented in one or more circuits or logic elements.

The techniques of this disclosure may be implemented in a wide variety of devices or apparatuses, including a wireless handset, an integrated circuit (IC) or a set of ICs (e.g., a chip set). Various components, modules, or units are described in this disclosure to emphasize functional aspects of devices configured to perform the disclosed techniques, but do not necessarily require realization by different hardware units. Rather, as described above, various units may be combined in a hardware unit or provided by a collection of interoperative hardware units, including one or more processors as described above, in conjunction with suitable software and/or firmware.

Various examples of the disclosure have been described. These and other examples are within the scope of the following claims.

1. A method comprising:
storing plurality of reference orientation templates, each of the plurality of reference orientation templates associated with a respective one of a plurality of different orientations of a patient's body part;
receiving, by processing circuitry, outputs of a sensor, wherein the sensor is responsive to a gravitational field;
determining, by the processing circuitry and based on the outputs of the sensor, an orientation vector of the sensor relative to a gravitational vector of the gravitational field;
for each of the plurality of body part orientations, calculating, by the processing circuitry, an angular distance metric between the orientation vector and each of the reference orientation templates;
determining, by the processing circuitry, which of the calculated angular distance metrics indicates the associated reference orientation template is closest to the orientation vector;
selecting, by the processing circuitry, the one of the plurality of reference orientation templates associated with the closest angular distance metric as the current orientation of the patient's body part.

2. The method of claim 1, wherein the calculated angular distance metric indicates its associated reference orientation template is closest to the orientation vector is the is the calculated angular distance metric with a minimum magnitude compared to the plurality of calculated angular distance metrics.

3. The method of claim 1, wherein the sensor is a three-axis accelerometer and the orientation vector comprises a respective value for each of axis of the accelerometer.

4. The method of claim 1, wherein the orientation of the patient's body part is an orientation of the patient's chest and wherein a total number of reference orientation templates in the plurality of reference orientation templates is five, wherein the five reference templates comprise orientations of the patient's chest as: upright, right side up, left side up, face down, face up.

5. The method of claim 1, wherein the orientation of the patient's body part is an orientation of the patient's chest and wherein a total number of reference orientation templates in the plurality of reference orientation templates is ten, wherein the ten reference orientation templates comprise orientations of the patient's chest as: upright, sitting forward, sitting back, sitting left side, sitting right side, lying left side, lying right side, lying face down, lying face up, and upside down.

6. The method of claim 1, wherein the orientation of the patient's body part is an orientation of the patient's chest and wherein:
the plurality of reference templates defines a set of sleeping angles,
the plurality of reference templates comprises a set of reference templates indicating the orientation of the patient's chest including: −30, −20, −10, 0, 10, 20, 30, 40, 50 and 60 degrees.

7. The method of claim 1, further comprising:
transmitting, by the processing circuitry and to an external computing device, the orientation vector of the sensor, based on the outputs of the sensor, wherein the patient's body part is in one or more predetermined reference orientation or orientations;
receiving, by the processing circuitry, a plurality of generated reference templates from the external computing device; and
storing, by the processing circuitry, the plurality of reference orientation templates at a memory location operatively coupled to the processing circuitry.

8. The method of claim 7, wherein the processing circuitry receives the plurality of generated reference orientation templates based on transmitting a single orientation vector to the external computing device.

9. A method comprising:
storing a first reference orientation template and a second reference orientation template for determining a value representative of a body part angle of a patient, the first reference template orthogonal to the second reference template;
receiving, by processing circuitry, outputs of a sensor, wherein the sensor is responsive to a gravitational field;
determining, by the processing circuitry and based on the outputs of the sensor, an orientation vector of the sensor relative to a gravitational vector of the gravitational field;
selecting, by the processing circuitry, one of the first reference template or the second reference template for determining the orientation value based on the orientation vector; and
determining, by the processing circuitry, the value representative of the body part angle of the patient based on a dot product of the orientation vector and the selected one of the first reference template or the second reference template.

10. The method of claim 9, wherein selecting one of the first reference orientation template or the second reference orientation template comprises:
determining, by the processing circuitry, whether a difference between a direction of the orientation vector and a direction of the first reference template satisfies a threshold, based on a first dot product;
in response to determining that the difference satisfies the threshold, selecting, by the processing circuitry, the second reference template.

11. The method of claim 9,
wherein the first reference template is associated with an upright orientation of the patient's body part;
wherein the second reference template is associated with a supine orientation of the patient's body part; and
wherein the value indicative of the body part angle is a value indicative of an angle relative to a central axis of the patient's body part relative to earth's gravity vector.

12. The method of claim 9, wherein the dot product is a first dot product and the body part angle is a first body part angle, the method further comprising:
storing a third reference template, wherein the third reference template is orthogonal to both the first reference orientation template and the second reference orientation template;
calculating, by the processing circuitry, a second dot product of the orientation vector and the third reference orientation template;
determining, by the processing circuitry, a second body part angle of the patient, wherein determining the second body part angle of the patient further comprises determining the second body part angle based on both the first dot product and the second dot product; and
wherein the second body part angle is a value indicative of an angle describing the body part orientation in terms of the patient's body part's rotation about an axis of the patient's body part.

13. The method of claim 9, wherein the processing circuitry is further configured to determine a category of position for the patient's body part based on category definitions involving ranges of values of the first body part angle and the second body part angle.

14. The method of claim 12,
wherein the third reference template is associated with a right-side or left-side up orientation of the patient's body part;
wherein the second reference template is associated with a supine orientation of the patient's body part; and
wherein the value indicative of the second body part angle is value indicative of a rotational side-angle.

15. The method of claim 9, further comprising:
transmitting, by the processing circuitry and to an external computing device, the orientation vector of the sensor, based on the outputs of the sensor, wherein the patient's body part is placed in a specific predetermined compensation reference orientation;
receiving, by the processing circuitry, a plurality of reference orientation templates generated by the external computing device: and
storing, by the processing circuitry, the plurality of reference orientation templates at a memory location operatively coupled to the processing circuitry.

16. The method of claim 15, wherein the processing circuitry receives the plurality of reference orientation templates based on transmitting a single orientation vector to the external computing device.

17. A system comprising:
a medical device, wherein the medical device is configured to be attached to a patient's body part in a fixed orientation relative to the patient's body part, the device comprising:
a sensor, wherein the sensor is responsive to a gravitational field;
processing circuitry operatively coupled to the sensor, wherein the processing circuitry is configured to:
receive outputs of the sensor;
calculate an orientation vector of the sensor, based on the outputs of the sensor, wherein a direction of the orientation vector is relative to the gravitational field;
store a first reference orientation template and a second reference orientation template for determining a value representative of an orientation of the patient's body part, the first reference orientation template orthogonal to the second reference orientation template;
select one of the first reference orientation template or the second reference orientation template for determining the value representative of a body part angle of the patient's body part based on the orientation vector; and
determine the value representative of the body part angle based on a dot product of the orientation vector and the selected one of the first reference orientation template or the second reference orientation template.

18. The system of claim 17, wherein the sensor is a three-axis accelerometer and the orientation vector comprises a respective value for each axis of the accelerometer.

19. The system of claim 17, wherein to select one of the first reference orientation template or the second reference orientation template for determining the orientation value based on the orientation vector comprises:
determine whether an angular difference between the orientation vector and a first reference orientation template satisfies a threshold, based on a dot product between the orientation vector and the first reference orientation;
in response to determining that the angular difference satisfies the threshold, select the second reference orientation template.

20. The system of claim 17,
wherein the first reference orientation template is associated with an upright orientation of the patient's body part;
wherein the second reference orientation template is associated with a supine orientation of the patient's body part; and
wherein the value indicative of the body part angle is value indicative of an angle relative to a central axis of the body part relative to earth's gravity vector.

21. The system of claim 17, wherein the dot product is a first dot product, wherein the processing circuitry is configured to:
store a third reference orientation template, wherein the third reference orientation template is orthogonal to both the first reference orientation template and the second reference orientation template
calculate a second dot product of the orientation vector and the third reference orientation template;
determine a second body part angle, wherein determining the second body part angle of the patient further comprises determining the second body part angle based on both the first dot product and the second dot product.

22. The system of claim 20, wherein the processing circuitry is further configured to determine a category of position for the patient's body part based on category definitions involving ranges of values of the first body part angle and the second body part angle.

23. The system of claim 20,
wherein the third reference orientation template is associated with a right-side up or left side up orientation of the patient's body part;
wherein the second reference template is associated with a supine orientation of the patient's body part; and
wherein the second body part angle is a value indicative of an angle describing the body part orientation in terms of a rotation of the body part about an axis of the body part.

24. The system of claim 17, further comprising an external computing device, wherein the processing circuitry is further configured to:
transmit to the external computing device the orientation vector of the sensor, based on the outputs of the sensor, wherein the patient's body part is in a predetermined reference orientation;
receive a plurality of reference orientation templates from an external computing device; and
store the plurality of reference orientation templates at a memory location operatively coupled to the processing circuitry.

25. The system of claim 24, wherein the processing circuitry receives the plurality of reference orientation templates based on transmitting a single orientation vector to the external computing device.

26. The system of claim 24, wherein the processing circuitry is further configured to output to the external computing device a history of patient body part orientation and body part position values, such that the external computing device converts the history of orientation and position values to a chart used to track and present patient body part orientation and position.

27. The system of claim 17, wherein the outputs of the sensor are a first set of outputs, wherein the processing circuitry is further configured to:
wait to determine the value representative of the body part angle until the processing circuitry determines a change in orientation;
in response to the change in orientation, receive a second set of outputs from the sensor;
update the value representative of the body part angle of the patient, based on the second set of outputs from the sensor.

28. The system of claim 27, wherein the processing circuitry is configured to carry out the measurement and calculation of body part orientation, after the processing circuitry detects a change in orientation as indicated by a threshold change in only one selected accelerometer axis or a change in one arithmetic combination of one, two or three axes of the sensor.

29. The system of claim 27, wherein the processing circuitry are configured so that the accelerometer axis or arithmetic combination of axes is selected to be the one of the set of axes that most closely aligns with the orientation vector.

30. The system of claim 17, further comprising an external computing device configured to:
receive a trend of dot products from the medical device;
convert the dot products to angle measurements;
output the converted angle measurements such that the trend of dot products is converted to derived activities of daily living in a manner to assist the patient and caregiver in understanding the health status of the patient.
